(19) United States
(12) Patent Application Publication
(10) Pub. No.: US 2018/0214440 A1
(43) Pub. Date: Aug. 2, 2018

(54) ORAL COMPOSITION

(71) Applicant: HELPERBY THERAPEUTICS LIMITED, London, Greater London (GB)

(72) Inventors: Anthony COATES, London (GB); Yanmin HU, London (GB); Sanju DHAWAN, Ahmedabad (IN); Kartik SHAH, Ahmedabad (IN)

(73) Assignee: HELPERBY THERAPEUTICS LIMITED, London, Greater London (GB)

(21) Appl. No.: 15/565,584

(22) PCT Filed: Apr. 11, 2016
(86) PCT No.: PCT/GB2016/051006

§ 371 Date: Apr. 19, 2018

(30) Foreign Application Priority Data
Apr. 11, 2015 (IN) 1523/MUM/2015
Sep. 7, 2015 (GB) 1515824.9

(51) Int. Cl. A61K 31/4745; A61Q 11/00; A61K 8/43; A61K 8/49; A61K 8/27; A61K 8/36; A61K 31/155; A61K 33/30; A61K 9/00; A61P 31/04

(52) U.S. Cl. CPC A61K 31/4745; A61Q 11/00; A61K 8/43; A61K 8/4926; A61P 31/04; A61K 8/36; A61K 31/155; A61K 33/30; A61K 9/006; A61K 8/27

(57) ABSTRACT

The invention provides an oral composition comprising 4-methyl-8-phenoxy-1-(2-phenylethyl)-2,3-dihydro-1H-pyrrolo[3,2-c]quinoline or a pharmaceutically acceptable salt and/or solvate thereof and chlorhexidine or a pharmaceutically acceptable salt and/or solvate thereof, wherein the composition is in the form of a mouthwash and comprises about 0.001% w/v to about 0.06% w/v of chlorhexidine or a pharmaceutically acceptable salt and/or solvate thereof. Also provided is an oral composition comprising 4-methyl-8-phenoxy-1-(2-phenylethyl)-2,3-dihydro-1H-pyrrolo[3,2-c]quinoline or a pharmaceutically acceptable salt and/or solvate thereof, chlorhexidine or a pharmaceutically acceptable salt and/or solvate thereof, and a zinc compound.

### FIELD OF THE INVENTION

This invention relates to an oral composition comprising a combination of antimicrobial agents and optionally a zinc compound for the prevention and/or treatment of microbial infections in the oral cavity. Preferably the oral composition is in the form of a mouthwash.

In particular, this invention relates to the use of 4-methyl-8-phenoxy-1-(2-phenylethyl)-2,3-dihydro-1H-pyrrolo[3,2-c]quinoline or a pharmaceutically acceptable salt and/or solvate thereof in combination with about 0.001% w/v to about 0.06% w/v of chlorhexidine or a pharmaceutically acceptable salt and/or solvate thereof in a mouthwash.

The invention also relates to the use of 4-methyl-8-phenoxy-1-(2-phenylethyl)-2,3-dihydro-1H-pyrrolo[3,2-c]quinoline or a pharmaceutically acceptable salt and/or solvate thereof in combination with chlorhexidine or a pharmaceutically acceptable salt and/or solvate thereof and a zinc compound in a mouthwash.

### BACKGROUND OF THE INVENTION

Antimicrobial agents have been widely used in preventive dentistry as inhibitors of plaque formation and/or of development of gingivitis. Chlorhexidine for example is commercially available, and is known to be effective against a wide range of Gram-positive and Gram-negative organisms found in the oral cavity. Chlorhexidine is consequently sold in Europe as an active ingredient of various mouthwashes.

One of these mouthwashes is Corsodyl® mouthwash. Corsodyl® mouthwash is manufactured by GlaxoSmithKline and is indicated for the inhibition of dental plaque, as an aid in the treatment and prevention of gingivitis, and in the maintenance of oral hygiene. The active ingredient of Corsodyl® mouthwash is 0.2% w/v chlorhexidine digluconate.

Chlorhexidine is also sold under the trade name Rivacol™. Rivacol™ chlorhexidine mouthwash is marketed by Perrigo Company plc and is indicated for the inhibition of dental p7laque, for the treatment and prevention of gingivitis and in maintaining oral hygiene. Like Corsodyl®, Rivacol™ includes 0.2% w/v chlorhexidine digluconate.

Continued or regular use of products containing such a high concentration of chlorhexidine is not, however, recommended. The side-effects from continued or regular use include staining of the teeth, the tongue and the gums, disturbances of taste sensations, burning sensation of the tongue, oral desquamation and swelling of the parotid glands.

A lower concentration of chlorhexidine digluconate (i.e. below 0.2% w/v) can therefore be found in various mouthwash products.

Colgate® PerioGard® mouthwash (manufactured by Colgate) for example includes 0.12% w/v chlorhexidine digluconate. This mouthwash is, however, still known to cause staining of teeth and the tongue, an alteration in taste perception, oral desquamation and swelling of the parotid glands.

Corsodyl® Daily mouthwash (manufactured by GlaxoSmithKline) includes 0.06% w/v chlorhexidine digluconate, and is recommended for daily use. Corsodyl® Daily is not reported to have the above-mentioned side effects (e.g. teeth staining), but is not indicated for the prevention or treatment of a microbial infection such as gingivitis. It is only recommended to remove dental plaque and maintain oral hygiene. It thus seems that at a concentration of 0.06% w/v, the antimicrobial activity of chlorhexidine is compromised.

CB12 mouthwash (manufactured by Meda Pharmaceuticals, UK) includes 0.025% w/v chlorhexidine diacetate along with 0.3% w/v zinc acetate, and is indicated for the prevention and treatment of bad breath. CB12 is not, however, indicated for the prevention or treatment of a microbial infection.

Formulations such as CB12 are disclosed in WO 0051559 (granted as European Patent 1156777B) which describes an oral composition for inhibiting oral malodor containing an antibacterial agent and a zinc compound. The composition is in the form of a mouthwash and contains 0.005-0.05% w/v of an antibacterial agent selected from bis-guanides and quaternary ammonium compounds, and 0.05-0.5% w/v of zinc acetate.

It can be seen from above that there is an unmet need for an effective oral composition which comprises a low concentration (e.g. about 0.001% w/v to about 0.06% w/v) of chlorhexidine or a pharmaceutically acceptable salt and/or solvate thereof for the treatment or prevention of microbial infections in the oral cavity.

### BRIEF DESCRIPTION OF THE DRAWINGS

FIG. 1 shows the time-kill curves for the dilutions of formulation (1);

FIG. 2 shows the time-kill curves for the dilutions of formulation (2);

FIG. 3 shows the time-kill curves for the dilutions of formulation (3);

FIG. 4 shows the time-kill curves for the chlorhexidine formulations CHX 0.06%, CHX 0.04%, CHX 0.03% and CHX 0.015%. These formulations included chlorhexidine digluconate at either 0.06% w/v, 0.04% w/v, 0.03% w/v or 0.015% w/v;

FIG. 5 shows the time-kill curves for the formulations F016A, F016B, F016C and F016D;

FIG. 6 shows the time-kill curves for Corsodyl® Daily Defence mouthwash 0.06% and a formulation solely containing 0.01% w/v HT61 HCl. Corsodyl® Daily Defence mouthwash 0.06% is a commercially available product manufactured by GlaxoSmithKline which contains 0.06% w/v chlorhexidine digluconate;

FIG. 7 shows the time-kill curve for the F021E formulation at 2-fold, 4-fold, 8-fold and 16-fold dilution. The F021E formulation is set out above in Table 3: it contains 0.01% w/v HT61 HCl, 0.06% w/v chlorhexidine gluconate and no surfactant (Cremophor® RH 40);

FIGS. 8 to 14 show the time-kill curves for the F021F, F021I, F021J, F021K, F021L, F021M and F021N formulations at 2-fold, 4-fold, 8-fold and 16-fold dilution;

FIG. 15 shows the time-kill curves for the F021F formulation and for Corsodyl® Daily diluted to a chlorhexidine concentration of 0.05% w/v;

FIG. 16 shows the time-kill curves for the F021I formulation and for Corsodyl® Daily diluted to a chlorhexidine concentration of 0.04% w/v;

FIG. 17 shows the time-kill curves for the F021J formulation and for Corsodyl® Daily diluted to a chlorhexidine concentration of 0.03% w/v;

FIG. 18 shows the time-kill curves for the F021K formulation and for Corsodyl® Daily diluted to a chlorhexidine concentration of 0.02% w/v;

FIG. 19 shows the time-kill curves for the F021L formulation and for Corsodyl® Daily diluted to a chlorhexidine concentration of 0.01% w/v;

FIG. 20 shows the time-kill curves for the F021L formulation and for CB12;

FIG. 21 contains the time-kill curves for each of the tested formulations against log phase S. aureus;

FIGS. 22, 23 and 24 show the selected ion flow tube mass spectrometry (SIFT-MS) results for H2S, volatile organic compounds (VOCs) and volatile sulphur compounds (VSCs) in the exhaled breath of each subject;

FIG. 25 shows results of an inhibition assay against S. aureus. Unbolded cells show growth inhibition. Bolded cells show growth.

FIG. 26 shows results of an inhibition assay against S. aureus. Unbolded cells show growth inhibition. Bolded cells show growth.

FIG. 27 shows results of an inhibition assay against S. aureus. Complete kill is represented by bolded cells.

FIG. 28 shows results of an inhibition assay against S. aureus. Complete kill is represented by bolded cells.

### DETAILED DESCRIPTION OF THE INVENTION

Two oral microbial infections which may be prevented or treated with antimicrobial agents are dental caries and periodontal disease. As with the prevention and treatment of other microbial infections, however, oral bacteria have responded to the use of antimicrobial agents by progressively gaining resistance to commonly used antibiotics (Sweeney 53(4), 567-576 (2004)).

One way of tackling the growing problem of resistant bacteria is the development of new classes of antimicrobial agents. However, until the introduction of linezolid in 2000, there had been no new class of antibiotic marketed for over 37 years. Moreover, even the development of new classes of antibiotic provides only a temporary solution, and indeed there have been reports of resistance of certain bacteria to linezolid (Lancet 357, 1179 (2001) and Lancet 358, 207-208 (2001)).

In order to develop more long-term solutions to the problem of bacterial resistance, it is clear that alternative approaches are required. It is also necessary to gain an understanding of the actual mechanisms by which bacteria generate resistance to antibiotic agents. To do this requires first a consideration of how current antibiotic agents work to kill bacteria.

International Patent Application, Publication Number WO2000028074 describes a method of screening compounds to determine their ability to kill clinically latent microorganisms. Using this method, it has been observed that many conventional antimicrobial agents, such as co-amoxiclav, azithromycin, levofloxacin, linezolid and mupirocin, which otherwise exhibit excellent biological activity against log phase (i.e. multiplying) bacteria, exhibit little or no activity against clinically latent microorganisms. This observation necessitated the development of novel antimicrobials which may be used to kill clinically latent microorganisms.

International Patent Application, Publication Numbers WO2007054693, WO2008117079 and WO2008142384 describe novel compounds which exhibit biological activity against clinically latent microorganisms. Examples of such compounds include 4-methyl-8-phenoxy-1-(2-phenylethyl)-2,3-dihydro-1H-pyrrolo[3,2-c]quinoline, 4-(3-benzylpyrrolidin-1-yl)-2-methyl-6-phenoxyquinoline, N-[4-(3-benzylpyrrolidin-1-yl)-2-methylquinolin-6-yl]benzamide and pharmaceutically acceptable salt and/or solvates thereof.

International Patent Application, Publication Number WO 2012017215 further describes a combination of 4-methyl-8-phenoxy-1-(2-phenylethyl)-2,3-dihydro-1H-pyrrolo[3,2-c]quinoline or a pharmaceutically acceptable salt and/or solvate thereof, and chlorhexidine or a pharmaceutically acceptable salt and/or solvate thereof, and reports synergistic antibacterial activity for this combination against clinically latent microorganisms.

The present invention is, however, based upon the unexpected finding that the antibacterial properties of chlorhexidine are maintained at a level of 0.06% w/v or below when the composition includes 4-methyl-8-phenoxy-1-(2-phenylethyl)-2,3-dihydro-1H-pyrrolo[3,2-c]quinoline or a pharmaceutically acceptable salt and/or solvate thereof. The oral compositions of the present invention are thus effective against a microbial infection even with a low concentration of about 0.001% w/v to about 0.06% w/v of chlorhexidine or pharmaceutically acceptable salt and/or solvate thereof.

The inventors have also surprisingly found that antibacterial activity is observed when 4-methyl-8-phenoxy-1-(2-phenylethyl)-2,3-dihydro-1H-pyrrolo[3,2-c]quinoline or a pharmaceutically acceptable salt and/or solvate thereof is combined with chlorhexidine or a pharmaceutically acceptable salt and/or solvate and a zinc compound in an oral composition.

This antibacterial activity advantageously provides a mouthwash which is able to prevent or treat a microbial infection even with a low concentration of chlorhexidine or a salt and/or solvate thereof. Compared to the commercially available products containing low concentrations of chlorhexidine (e.g. Corsodyl® Daily Defence Mouthwash and CB12), the inventors found that the combination of the present invention provides a surprisingly faster kill of microorganisms associated with a microbial infection.

The surprising antimicrobial activity of the compositions of the present invention thus offers the opportunity to provide improved mouthwash formulations without the problems mentioned above associated with high concentrations of chlorhexidine which effectively prevent or treat microbial infections.

In each embodiment of the invention, the antibacterial activity of the combined agents is preferably synergistic, i.e. greater than the expected additive effect of each agent at the stated dosage level.

Synergy in the context of antimicrobials drugs is measured in a number of ways that conform to the generally accepted opinion that “synergy” is an effect greater than additive. One of the ways to assess whether synergy has been observed is to use the “chequerboard” technique. This is a well-accepted method that leads to the generation of a value called the fractional inhibitory concentration index (FICI). Orhan et al J. Clin. Microbiol. 2005, 43(1):140 describes the chequerboard method and analysis in the paragraph bridging pages 140-141, and explains that the FICI value is a ratio of the sum of the MIC (Minimum Inhibitory Concentration) level of each individual component alone and in the mixture. The combination is considered synergistic when the ΣFIC is <0.5, indifferent when the ΣFIC is >0.5 to <2, and antagonistic when the ΣFIC is >2.

Another accepted test for ascertaining the presence or absence of synergy is to use time-kill methods where the dynamic effect of a drug combination is compared to each drug alone when assessing the effect on bacterial log or stationary-growth over time. Again, the possible results are for synergistic, additive or antagonistic effects.

In one embodiment the present invention provides an oral composition comprising 4-methyl-8-phenoxy-1-(2-phenylethyl)-2,3-dihydro-1H-pyrrolo[3,2-c]quinoline or a pharmaceutically acceptable salt and/or solvate thereof and chlorhexidine or a pharmaceutically acceptable salt and/or solvate thereof, where the composition is in the form of a mouthwash and comprises about 0.001% w/v to about 0.06% w/v of the chlorhexidine or pharmaceutically acceptable salt and/or solvate thereof.

In a further embodiment, the present invention provides an oral composition comprising 4-methyl-8-phenoxy-1-(2-phenylethyl)-2,3-dihydro-1H-pyrrolo[3,2-c]quinoline or a pharmaceutically acceptable salt and/or solvate thereof, chlorhexidine or a pharmaceutically acceptable salt and/or solvate thereof, and a zinc compound, where the composition is in the form of a mouthwash.

In another embodiment, the present invention provides the herein defined oral compositions for use in the prevention and/or treatment of a microbial infection.

In another embodiment, the present invention provides the herein defined oral compositions for use in killing clinically latent microorganisms associated with a microbial infection. Preferably a microbial infection of the oral cavity.

In a further embodiment, the present invention provides the use of 4-methyl-8-phenoxy-1-(2-phenylethyl)-2,3-dihydro-1H-pyrrolo[3,2-c]quinoline or a pharmaceutically acceptable salt and/or solvate thereof in combination with about 0.001% w/v to about 0.06% w/v of chlorhexidine or a pharmaceutically acceptable salt and/or solvate thereof for the manufacture of a medicament for the prevention and/or treatment of a microbial infection; in particular for killing multiplying, non-multiplying and/or clinically latent microorganisms associated with such an infection.

In a further embodiment, the present invention provides the use of 4-methyl-8-phenoxy-1-(2-phenylethyl)-2,3-dihydro-1H-pyrrolo[3,2-c]quinoline or a pharmaceutically acceptable salt and/or solvate thereof in combination with about 0.001% w/v to about 0.06% w/v of chlorhexidine or a pharmaceutically acceptable salt and/or solvate thereof for the prevention and/or treatment of a microbial infection; in particular for killing multiplying, non-multiplying and/or clinically latent microorganisms associated with such an infection.

In a further embodiment, the present invention provides the use of 4-methyl-8-phenoxy-1-(2-phenylethyl)-2,3-dihydro-1H-pyrrolo[3,2-c]quinoline or a pharmaceutically acceptable salt and/or solvate thereof in combination with chlorhexidine or a pharmaceutically acceptable salt and/or solvate thereof and a zinc compound for the manufacture of a medicament for the prevention and/or treatment of a microbial infection; in particular for killing multiplying, non-multiplying and/or clinically latent microorganisms associated with such an infection.

In a further embodiment, the present invention provides the use of 4-methyl-8-phenoxy-1-(2-phenylethyl)-2,3-dihydro-1H-pyrrolo[3,2-c]quinoline or a pharmaceutically acceptable salt and/or solvate thereof in combination with chlorhexidine or a pharmaceutically acceptable salt and/or solvate thereof and a zinc compound for the prevention and/or treatment of a microbial infection; in particular for killing multiplying, non-multiplying and/or clinically latent microorganisms associated with such an infection.

The invention further provides a method of preventing or treating a microbial infection, in particular killing multiplying, non-multiplying and/or clinically latent microorganisms associated with such an infection, which comprises administering to a mammal, including man, an oral composition as defined herein.

In another embodiment, the invention provides the use of 4-methyl-8-phenoxy-1-(2-phenylethyl)-2,3-dihydro-1H-pyrrolo[3,2-c]quinoline or a pharmaceutically acceptable salt and/or solvate thereof in combination with about 0.001% w/v to about 0.06% w/v of chlorhexidine or a pharmaceutically acceptable salt and/or solvate thereof as a mouthwash.

In another embodiment, the invention provides the use of 4-methyl-8-phenoxy-1-(2-phenylethyl)-2,3-dihydro-1H-pyrrolo[3,2-c]quinoline or a pharmaceutically acceptable salt and/or solvate thereof in combination with chlorhexidine or a pharmaceutically acceptable salt and/or solvate thereof and a zinc compound as a mouthwash.

In another embodiment, the invention provides a mouthwash composition containing 0.025% w/v or less chlorhexidine which exhibits antimicrobial activity comparable to a mouthwash containing 0.06% w/v chlorhexidine (e.g. Corsodyl™ Daily Mouthwash). The mouthwash composition in this embodiment has prophylactic and/or therapeutic activity equivalent to 0.06% w/v with lower amounts of chlorhexidine. The composition contains 0.025% w/v or less chlorhexidine and HT61 (4-methyl-8-phenoxy-1-(2-phenylethyl)-2,3-dihydro-1H-pyrrolo[3,2-c]quinoline or a pharmaceutically acceptable salt, solvate, derivative, enantiomer or mixture thereof) and provides antimicrobial activity equivalent to 0.06% w/v chlorhexidine (Corsodyl™ Daily Mouthwash), when the composition is free from surfactant.

An object of the present invention is to provide a mouthwash composition with reduced side effects such as dental staining, foul and bitter taste associated with chlorhexidine.

As used herein, the terms “combination” and “in combination with” refer to both separate and sequential administration of 4-methyl-8-phenoxy-1-(2-phenylethyl)-2,3-dihydro-1H-pyrrolo[3,2-c]quinoline or a pharmaceutically acceptable salt and/or solvate thereof, chlorhexidine or a pharmaceutically acceptable salt and/or solvate thereof and/or a zinc compound.

When the agents are administered sequentially, either 4-methyl-8-phenoxy-1-(2-phenylethyl)-2,3-dihydro-1H-pyrrolo[3,2-c]quinoline or a pharmaceutically acceptable salt and/or solvate thereof, or chlorhexidine or a pharmaceutically acceptable salt and/or solvate thereof or the zinc compound (when present) may be administered first. When administration is simultaneous, the agents may be administered either in the same or a different pharmaceutical composition. Adjunctive therapy, i.e. where one agent is used as a primary treatment and the other agent is used to assist that primary treatment, is also an embodiment of the present invention.

According to a further embodiment of the invention, there is provided a product comprising 4-methyl-8-phenoxy-1-(2-phenylethyl)-2,3-dihydro-1H-pyrrolo[3,2-c]quinoline or a pharmaceutically acceptable salt and/or solvate thereof and about 0.001% w/v to about 0.06% w/v of chlorhexidine or a pharmaceutically acceptable salt and/or solvate thereof as a combined preparation for simultaneous, separate or sequential use in the prevention and/or treatment of a microbial infection.

There is also provided a product comprising 4-methyl-8-phenoxy-1-(2-phenylethyl)-2,3-dihydro-1H-pyrrolo[3,2-c]quinoline or a pharmaceutically acceptable salt and/or solvate thereof, chlorhexidine or a pharmaceutically acceptable salt and/or solvate thereof and a zinc compound as a combined preparation for simultaneous, separate or sequential use in the prevention and/or treatment of a microbial infection.

There is also provided a pharmaceutical composition comprising 4-methyl-8-phenoxy-1-(2-phenylethyl)-2,3-dihydro-1H-pyrrolo[3,2-c]quinoline or a pharmaceutically acceptable salt and/or solvate and/or solvate thereof, and about 0.001% w/v to about 0.06% w/v of chlorhexidine or a pharmaceutically acceptable salt and/or solvate thereof, and a pharmaceutically acceptable adjuvant, diluent or carrier. Alternatively a pharmaceutical composition is provided which comprises 4-methyl-8-phenoxy-1-(2-phenylethyl)-2,3-dihydro-1H-pyrrolo[3,2-c]quinoline or a pharmaceutically acceptable salt and/or solvate thereof, chlorhexidine or a pharmaceutically acceptable salt and/or solvate thereof, a zinc compound, and a pharmaceutically acceptable adjuvant, diluent or carrier.

Such pharmaceutical compositions may be used for the prevention and/or treatment of microbial infections, and in particular for use in killing multiplying, non-multiplying and/or clinically latent microorganisms associated with a microbial infection.

The oral compositions of the present invention may be used to prevent and/or treat microbial infections. In particular they may be used to kill multiplying, non-multiplying and/or clinically latent microorganisms associated with microbial infections. References herein to the treatment of a microbial infection therefore include killing multiplying, non-multiplying and/or clinically latent microorganisms associated with such infections.

As used herein, “kill” means a loss of viability as assessed by a lack of metabolic activity.

As used herein, “clinically latent microorganism” means a microorganism that is metabolically active but has a growth rate that is below the threshold of infectious disease expression. The threshold of infectious disease expression refers to the growth rate threshold below which symptoms of infectious disease in a host are absent.

The metabolic activity of clinically latent microorganisms can be determined by several methods known to those skilled in the art; for example, by measuring mRNA levels in the microorganisms or by determining their rate of uridine uptake. In this respect, clinically latent microorganisms, when compared to microorganisms under logarithmic growth conditions (in vitro or in vivo), possess reduced but still significant levels of:
(I) mRNA (e.g. from 0.0001 to 50%, such as from 1 to 30, 5 to 25 or 10 to 20%, of the level of mRNA); and/or
(II) uridine (e.g. [³H]uridine) uptake (e.g. from 0.0005 to 50%, such as from 1 to 40, 15 to 35 or 20 to 30% of the level of [³H]uridine uptake).

Clinically latent microorganisms typically possess a number of identifiable characteristics. For example, they may be viable but non-culturable; i.e. they cannot typically be detected by standard culture techniques, but are detectable and quantifiable by techniques such as broth dilution counting, microscopy, or molecular techniques such as polymerase chain reaction. In addition, clinically latent microorganisms are phenotypically tolerant, and as such are sensitive (in log phase) to the biostatic effects of conventional antimicrobial agents (i.e. microorganisms for which the minimum inhibitory concentration (MIC) of a conventional antimicrobial is substantially unchanged); but possess drastically decreased susceptibility to drug-induced killing (e.g. microorganisms for which, with any given conventional antimicrobial agent, the ratio of minimum microbiocidal concentration (e.g. minimum bactericidal concentration, MBC) to MIC is 10 or more).

As used herein, the term “microorganisms” means fungi and bacteria. References herein to “microbial”, “antimicrobial” and “antimicrobially” shall be interpreted accordingly. For example, the term “microbial” means fungal or bacterial, and “microbial infection” means any fungal or bacterial infection.

As used herein, the term “bacteria” (and derivatives thereof, such as “microbial infection”) includes, but is not limited to, references to organisms (or infections due to organisms) of the following classes and specific types:

Gram-positive cocci, such as Staphylococci (e.g. Staph. aureus, Staph. epidermidis, Staph. saprophyticus, Staph. auricularis, Staph. capitis capitis, Staph. c. ureolyticus, Staph. caprae, Staph. cohnii cohnii, Staph. c. urealyticus, Staph. equorum, Staph. gallinarum, Staph. haemolyticus, Staph. hominis hominis, Staph. h. novobiosepticius, Staph. hyicus, Staph. intermedius, Staph. lugdunensis, Staph. pasteuri, Staph. saccharolyticus, Staph. schleiferi schleiferi, Staph. s. coagulans, Staph. sciuri, Staph. simulans, Staph. warneri and Staph. xylosus);

Streptococci (e.g. beta-haemolytic, pyogenic streptococci (such as Strept. agalactiae, Strept. canis, Strept. dysgalactiae dysgalactiae, Strept. dysgalactiae equisimilis, Strept. equi equi, Strept. equi zooepidemicus, Strept. iniae, Strept. porcinus and Strept. pyogenes),

microaerophilic, pyogenic streptococci (Streptococcus “milleri”, such as Strept. anginosus, Strept. constellatus constellatus, Strept. constellatus pharyngidis and Strept. intermedius), oral streptococci of the “mitis” (alpha-haemolytic-Streptococcus “viridans”, such as Strept. mitis, Strept. oralis, Strept. sanguinis, Strept. cristatus, Strept. gordonii and Strept. parasanguinis), “salivarius” (non-haemolytic, such as Strept. salivarius and Strept. vestibularis) and “mutans” (tooth-surface streptococci, such as Strept. criceti, Strept. mutans, Strept. ratti and Strept. sobrinus) groups, Strept. acidominimus, Strept. bovis, Strept. faecalis, Strept. equinus, Strept. pneumoniae and Strept. suis, or Streptococci alternatively classified as Group A, B, C, D, E, G, L, P, U or V Streptococcus).

Other Gram-positive bacteria including:

Lactobacillus; Micrococcus; Rothia dentocariosa; Peptococcus (e.g. Peptococcus niger); Peptostreptococcus; Arachnia propionica (Propionibacterium propionicus); Solobacterium moorei; and Corynebacterium.

Gram-negative cocci, mainly Gram-negative anaerobes. Such Gram-negative cocci include:

Enterobacteriaceae, such as Escherichia coli, Enterobacter (e.g. and Enterobacter cloacae); Enterococci (e.g. Enterococcus faecalis, and Enterococcus faecium); Eubacterium; Pseudomonas (e.g. Ps. aeruginosa, Ps. maltophilia (Stenotrophomonas maltophilia), Ps. alcaligenes, Ps. chlororaphis, Ps. fluorescens, Ps. luteola. Ps. mendocina, Ps. monteilii, Ps. oryzihabitans, Ps. pertocinogena, Ps. pseudalcaligenes, Ps. putida and Ps. stutzen);

Haemophilus parainfuenzae and Haemophilus paraphrophilus; Leptotrichia buccalis; Mycoplasma;

Bacteroides (e.g. Bacteroides fragilis; Bacteroides gingivalis; Bacteroides intermedius, Bacteroides melaninogenicus; and Bacteroides loescheii);

Aggregatibacter actinomycetemcomitans; Buchnera aphidicola;

Campylobacter (e.g. Campylobacter coli; Campylobacter sputorum; and Campylobacter upsaliensis); Eikenella corrodens;

Actinobacillus (e.g. Actinobacillus actinomycetemcomitans, Actinobacillus hominis, and Actinobacillus lignieresii);

Actinomyces (e.g. Actinomyces israelii, Actinomyces viscosus, and Actinomyces naeslundii));

Treponema (Treponema refringens, Treponema denticola);

Tannerella forsythia;

Veillonella;

Centipeda periodontii;

Flavobacteriaceae, such as Capnocytophaga (e.g. Capnocytophaga canimorsus, Capnocytophaga cynodegmi, Capnocytophaga gingivalis, Capnocytophaga granulosa, Capnocytophaga haemolytica, Capnocytophaga ochracea and Capnocytophaga sputigena); Porphyromonas (e.g. Porphyromonas asaccharolytica, Porphyromonas cangingivalis, Porphyromonas canoris, Porphyromonas cansulci, Porphyromonas catoniae, Porphyromonas circumdentaria, Porphyromonas crevioricanis, Porphyromonas endodontalis, Porphyromonas gingivalis, Porphyromonas gingivicanis, Porphyromonas levii and Porphyromonas macacae);

Fusobacterium (e.g. F. nucleatum nucleatum, F. nucleatum fusiforme, F. nucleatum polymorphum, F. nucleatum vincentii, and F. periodonticum);

Vibrio sputorum;

Prevotella (e.g. Prevotella melaninogenica and Prevotella intermedia);

Wolinella succinogenes; and

Gemella (e.g. Gemella bergeri, Gemella haemolysans, Gemella morbillorum and Gemella sanguinis).

As used herein, the term “fungi” (and derivatives thereof, such as “fungal infection”) includes, but is not limited to, references to organisms (or infections due to organisms) of the following classes and specific types:

Candida (e.g. Candida albicans, Candida glabrata, Candida guilliermondii, Candida krusei, Candida parapsilosis, Candida tropicalis and Candida pelliculosa); and

Torulopsis glabrata.

Particular bacteria that may treated using an oral composition of the invention include:

Staphylococci, such as Staph. aureus (either Methicillin-sensitive (i.e. MSSA) or Methicillin-resistant (i.e. MRSA)) and Staph. epidermidis; Bacteroides, such as Bacteroides loescheii; Centipeda periodontii; Eikenella corrodens; Enterobacteriaceae; Fusobacterium nucleatum nucleatum; Fusobacterium nucleatum polymorphum; Fusobacterium nucleatum vincentii; Fusobacterium periodonticum; Porphyromonas endodontalis; Porphyromonas gingivalis; Prevotella melaninogenica; Prevotella intermedia; Solobacterium moorei; Tannerella forsythia; and Treponema denticola.

Preferably the bacterium is Staphylococci. More preferably the bacterium is Staphylococci aureus.

Particular fungi that may be treated using the oral compositions of the invention include Candida, e.g. Candida albicans.

Particular conditions which may be prevented and/or treated using the combinations of the present invention include abscesses, actinomycosis, bleeding of the gums, calculus, dental caries, gingivitis, infections following dental operations, infections in the oral region, mouth odour, periodontal disease, plaque, systemic infections, tonsillitis, or infections with or caused by any of the above-mentioned bacteria. For example, infections caused by Staph. aureus.

References herein to “treatment” extend to prophylaxis as well as the treatment of established diseases or symptoms.

Generally it is recommended to use the oral composition as a mouthwash one, two or three times daily for at least 30 seconds. Preferably the oral composition is used as a mouthwash once daily for at least 30 seconds.

As used herein the term “pharmaceutically acceptable salt and/solvate thereof” means:

(a) pharmaceutically acceptable salts; and/or

(b) solvates (including hydrates).

Suitable acid addition salts include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulfonate salts (e.g. benzenesulfonate, methyl-, bromo- or chloro-benzenesulfonate, xylenesulfonate, methanesulfonate, ethanesulfonate, propanesulfonate, hydroxyethanesulfonate, 1- or 2-naphthalene-sulfonate or 1,5-naphthalenedisulfonate salts) or sulfate, pyrosulfate, bisulfate, sulfite, bisulfite, phosphate, monohydrogenphosphate, dihydrogenphosphate, metaphosphate, pyrophosphate or nitrate salts, and the like.

For the avoidance of doubt, references herein to 4-methyl-8-phenoxy-1-(2-phenylethyl)-2,3-dihydro-1H-pyrrolo[3,2-c]quinoline or HT61 mean a compound having the following chemical structure:

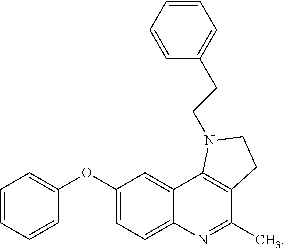 [SMILES: Cc1nc2ccc(Oc3ccccc3)cc2c2c1CCN2CCc1ccccc1]

4-methyl-8-phenoxy-1-(2-phenylethyl)-2,3-dihydro-1H-pyrrolo[3,2-c]quinoline or a pharmaceutically acceptable salt and/or solvate thereof may be prepared by methods known in the art, for example by following the methods disclosed in International Patent Application, Publication Numbers WO2007054693 and WO2008056151.

Preferred pharmaceutically acceptable salts of 4-methyl-8-phenoxy-1-(2-phenylethyl)-2,3-dihydro-1H-pyrrolo[3,2-c]quinoline include the hydrochloride and mesylate salt.

Chlorhexidine is a cationic polybiguanide also known as N,N′″1,6-Hexanediylbis[N′-(4-chlorophenyl)(imidodicarbonimidic diamide)] with the chemical formula:

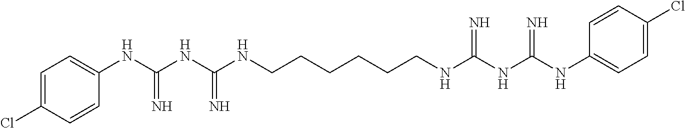 [SMILES: N=C(NCCCCCCNC(=N)NC(=N)Nc1ccc(Cl)cc1)NC(=N)Nc1ccc(Cl)cc1]

Chlorhexidine and its pharmaceutically acceptable salts and/or solvates are commercially available, for example from Sigma Aldrich Limited. Preferred pharmaceutically acceptable salts of chlorhexidine include the hydrochloride, dihydrochloride, diacetate, acetate, digluconate and gluconate salts thereof. Particularly preferred salts are the diacetate and digluconate salts, especially chlorhexidine digluconate.

The oral compositions of the invention may include a zinc compound. Preferably the zinc compound is a zinc salt such as zinc acetate.

The oral compositions of the invention are preferably in the form of a mouthwash. The composition may also therefore include an orally acceptable carrier or diluent. The carrier or diluent may be a substance which is typically used for oral hygiene compositions such as water or aqueous alcohol (e.g. aqueous ethanol).

In one embodiment the oral compositions of the invention do not contain any alcohol, i.e. the mouthwash is alcohol-free.

In one embodiment the oral compositions of the invention are aqueous solutions comprising 4-methyl-8-phenoxy-1-(2-phenylethyl)-2,3-dihydro-1H-pyrrolo[3,2-c]quinoline or a pharmaceutically acceptable salt and/or solvate thereof and chlorhexidine or a pharmaceutically acceptable salt and/or solvate thereof and optionally a zinc compound.

The oral compositions of the invention may also include one or more additional ingredients which are typically used for oral hygiene compositions. Such additional ingredients are known to the person skilled in the art and would be selected so as to be compatible with the other components of the oral compositions disclosed herein.

Examples of additional ingredients include other active ingredients conventionally used in oral hygiene compositions such as benzydamine, betamethasone, cetylpyridinium chloride, hexetidine, benzoic acid, liniment, sodium perborate, methyl salicylate, triclosan, benzalkonium chloride, methylparaben, hydrogen peroxide, domiphen bromide, sanguinarine, sodium bicarbonate, sodium chloride, sodium lauryl sulfate, tetracycline, tranexamic acid and fluoride.

Preferably the compositions of the invention include a fluoride such as an alkali metal or amine fluoride salt, e.g. sodium fluoride.

The oral compositions of the invention may also include one or more additional ingredients selected from the group consisting of essential oils, thickening agents, buffering agents, colouring agents, flavouring agents, sweetening agents, and preservatives. Suitable essential oils, thickening agents, buffering agents, colouring agents, flavouring agents, sweetening agents and preservatives would be known to the person skilled in the art.

For example, the essential oil may include phenol, thymol, eugenol, eucalyptol and/or menthol. The thickening agent may include cellulose gum, hydroxyethylcellulose, hydroxypropyl methylcellulose, glycerine/glycerine, sodium methyl cocoyl taurate, polyvinylpyrrolidone, propylene glycol, propylene glycol alginate, tetrapotassium pyrophosphate/tetrasodium pyrophosphate, titanium dioxide and/or cocamidopropyl betaine. The buffering agent may include sodium citrate, benzoic acid, sodium bicarbonate, sodium dodecyl sulfate, phosphate buffer saline, pentasodium triphosphate and/or citric acid. The colouring agent may include CI 74160, CI 15985, CI 18965, CI 18965, CI 42051, CI 42053, CI 42090, CI 73360, CI 77891, CI 19140 and/or CI 17200, where CI stands for Colour Index. The flavouring agent may include any aroma compound, eucalyptol, propylparaben, peppermint, menthol, methyl salicylate, anethole, viridis mint, limonene, cinnamaldehyde, and/or eugenol. The sweetening agent may include acesulfame potassium, stevia extract, neotame, aspartame, saccharin, sorbitol, sucralose, sodium saccharin and/or xylitol. The preservative may include sodium benzoate, methylisothiazolinone, methylparaben, benzoic acid, benzyl alcohol, citric acid, potassium sorbate, propylparaben, sodium phosphate and/or triclosan.

It can be seen that some additional ingredients fall under multiple categories, for example menthol is an essential oil and a flavouring agent. The skilled person would, however, know how to formulate an oral composition suitable for use as a mouthwash regardless of whether e.g. menthol was included as an essential, a flavouring agent or both.

The mouthwash composition of the invention may also include pharmaceutically acceptable excipients including solvents, co-solvents, viscosity enhancers, preservatives, sweeteners, flavours, colours or mixtures thereof. The solvents/co-solvent/viscosity enhancer may be selected from water, glycerine, propylene glycol, polyethylene glycol, sorbitol, alcohol, liquid glucose or combination thereof. The sweeteners may be selected from sucralose, neotame, aspartame, assesulfam, potassium and combination thereof. Preservatives may be selected from methyl paraben, propyl paraben, sodium benzoate, propyl gallate, benzyl alcohol, BKC and combination thereof.

The oral compositions of the present invention may also include a surfactant. Suitable surfactants would be known to the person skilled in the art and would be selected so as to be compatible with the other components of the oral composition as disclosed herein.

The surfactant may include hydrogenated caster oil, polyethylene glycol (e.g. PEG-12, PEG-180, PEG-6 and PEG-32), polyethylene glycol/hydrogenated caster oil (e.g. PEG-40 or PEG-60), Poloxamer 407, Polysorbate 20, polyoxyethylene fatty acid esters, polyethoxylated sorbitol esters (e.g. products marketed under the trade name Tween® by Croda), polycondensates of ethylene oxide and propylene oxide (poloxamers such as those marketed under the trade name Pluronic® by BASF), condensates of propylene glycol, polyethoxylated hydrogenated castor oil (e.g. Cremophor® such as those marketed by BASF including Cremophor® RH 40) and sorbitan fatty esters.

Although surfactants are typically found in oral compositions of the type described, it has been observed that a surfactant is not always required if a flavouring agent having a high water solubility is used, the surfactant can be omitted. This is a significant observation as the presence of a surfactant is associated with a masking of the anti-bacterial effect desired of the composition.

Surprisingly it was found that antimicrobial activity of HT61 reduces significantly in the presence of a surfactant. It was surprisingly found that addition of surfactant to the formulation containing chlorhexidine and HT61 reduces the synergistic effect. Similar results were obtained when mouthwash compositions were prepared with and without surfactant. Formulation without surfactant showed better antimicrobial activity compared to formulation with surfactant containing

Therefore, in one embodiment the composition does not include a surfactant. For example, in one embodiment the composition does not include a polyethoxylated hydrogenated castor oil such as Cremophor® RH 40. Preferably, when the composition does not contain a surfactant, a flavouring agent having a high water solubility is included in the composition. For example, the flavouring agent may have a water solubility of about 1.9 g per 100 ml at 20° C.

The oral compositions of the invention will have a pH which is orally acceptable, typically ranging from about pH 4 to 10, for example between 5 and 8.

The oral compositions may conveniently be presented in unit dosage form and may be prepared by any of the methods well known in the art of pharmacy e.g. as described in “Remington: The Science and Practice of Pharmacy”, Lippincott Williams and Wilkins, 21^{st} Edition, (2005). Suitable methods include the step of bringing into association the active ingredients with a carrier which constitutes one or more additional ingredients. In general, compositions are prepared by uniformly and intimately bringing into association the active ingredients with liquid carriers and then, if necessary, formulating the product into the desired composition. For example, the oral compositions may be prepared by admixing the ingredients in the appropriate relative amounts in any order that is convenient and thereafter and if necessary, adjusting the pH to give a final value within the above-mentioned ranges.

When formulated with additional ingredients, the active ingredients (4-methyl-8-phenoxy-1-(2-phenylethyl)-2,3-dihydro-1H-pyrrolo[3,2-c]quinoline or a pharmaceutically acceptable salt and/or solvate thereof, chlorhexidine or a pharmaceutically acceptable salt and/or solvate thereof and optionally a zinc compound) may be present in a concentration from 0.1 to 99.5% w/v (such as from 0.5 to 95%) of the total oral composition; conveniently from 0.01 to 50%, preferably from 0.01 to 1%, more preferably from 0.01 to 0.5% w/v of the total oral composition.

In one embodiment the chlorhexidine or a pharmaceutically acceptable salt and/or solvate thereof is included in the oral composition at about 0.001 to about 0.06% w/v, more preferably from about 0.001 to about 0.05% w/v, particularly preferably from about 0.01 to about 0.05% w/v for example 0.001, 0.002, 0.003, 0.004, 0.005, 0.006, 0.007, 0.008, 0.009, 0.01, 0.015, 0.02, 0.025, 0.03, 0.04, 0.05 or 0.06% w/v of the oral composition.

The percentage w/v (% w/v) of chlorhexidine or a pharmaceutically acceptable salt and/or solvate thereof is calculated based on the weight of the chlorhexidine or chlorhexidine salt per se in the oral composition. For example “0.06% w/v chlorhexidine digluconate” means that there is 0.06% of the chlorhexidine digluconate in the oral composition based on the weight of the salt and the volume of the overall composition.

A suitable concentration for 4-methyl-8-phenoxy-1-(2-phenylethyl)-2,3-dihydro-1H-pyrrolo[3,2-c]quinoline or a pharmaceutically acceptable salt and/or solvate thereof is from about 0.001 to about 0.5% w/v, preferably from about 0.005 to about 0.05% w/v, more preferably from about 0.005 to about 0.03% w/v, for example 0.001, 0.002, 0.0025, 0.003, 0.005, 0.075, 0.01, 0.02, 0.03, 0.04 or 0.05% w/v of the oral composition.

In one embodiment the oral composition comprises about 0.001% w/v to about 0.06% w/v of chlorhexidine or a pharmaceutically acceptable salt and/or solvate thereof and from about 0.005 to about 0.03% w/v of 4-methyl-8-phenoxy-1-(2-phenylethyl)-2,3-dihydro-1H-pyrrolo[3,2-c]quinoline or a pharmaceutically acceptable salt and/or solvate thereof. Preferably the oral composition comprises from about 0.001 to about 0.06% w/v of chlorhexidine or a pharmaceutically acceptable salt and/or solvate thereof, more preferably from about 0.01 to 0.06% w/v of the oral composition.

In one embodiment the oral composition comprises about 0.05% w/v chlorhexidine or a pharmaceutically acceptable salt and/or solvate thereof in combination with about 0.02% w/v 4-methyl-8-phenoxy-1-(2-phenylethyl)-2,3-dihydro-1H-pyrrolo[3,2-c]quinoline or a pharmaceutically acceptable salt and/or solvate thereof.

In another embodiment the oral composition comprises about 0.01% w/v chlorhexidine or a pharmaceutically acceptable salt and/or solvate thereof in combination with about 0.01% w/v 4-methyl-8-phenoxy-1-(2-phenylethyl)-2,3-dihydro-1H-pyrrolo[3,2-c]quinoline or a pharmaceutically acceptable salt and/or solvate thereof.

In a further embodiment the oral composition comprises about 0.03% w/v chlorhexidine or a pharmaceutically acceptable salt and/or solvate thereof in combination with about 0.01% w/v 4-methyl-8-phenoxy-1-(2-phenylethyl)-2,3-dihydro-1H-pyrrolo[3,2-c]quinoline or a pharmaceutically acceptable salt and/or solvate thereof.

A suitable concentration for the zinc compound, when included in the oral composition of the invention, is from about 0.01 to 0.5% w/v, preferably from about 0.01 to 0.3% w/v, more preferably from about 0.07 to 0.3%, for example, 0.01, 0.02, 0.03, 0.04, 0.05, 0.06, 0.07, 0.08, 0.09, 0.1, 0.2, 0.3, 0.4 or 0.5% w/v of the oral composition.

In one embodiment the oral composition includes about 0.03% w/v chlorhexidine or a pharmaceutically acceptable salt and/or solvate thereof in combination with about 0.03% w/v 4-methyl-8-phenoxy-1-(2-phenylethyl)-2,3-dihydro-1H-pyrrolo[3,2-c]quinoline or a pharmaceutically acceptable salt and/or solvate thereof and 0.30% w/v of a zinc compound.

The oral compositions may be prepared from discrete units such as capsules, sachets or tablets, each containing a predetermined amount of active ingredient; or from powder or granules.

A tablet may be made by compression or moulding, optionally with one or more excipients. Compressed tablets may be prepared by compressing in a suitable machine the active ingredient in a free-flowing form such as a powder or granules, optionally mixed with other conventional excipients such as binding agents (e.g. syrup, acacia, gelatin, sorbitol, tragacanth, mucilage of starch, polyvinylpyrrolidone and/or hydroxymethyl cellulose), fillers (e.g. lactose, sugar, microcrystalline cellulose, maize-starch, calcium phosphate and/or sorbitol), lubricants (e.g. magnesium stearate, stearic acid, talc, polyethylene glycol and/or silica), disintegrants (e.g. potato starch, croscarmellose sodium and/or sodium starch glycolate) and wetting agents (e.g. sodium lauryl sulphate). Moulded tablets may be made by moulding in a suitable machine a mixture of the powdered active ingredient with an inert liquid diluent. The tablets may be optionally coated or scored and may be formulated so as to provide controlled release (e.g. delayed, sustained, or pulsed release, or a combination of immediate release and controlled release) of the active ingredients.

Formulations containing the active ingredients may for instance also be presented as a dry product for constitution with water or another suitable vehicle (e.g. aqueous alcohol) before use. Such liquid preparations may contain conventional additives such as suspending agents (e.g. sorbitol syrup, methyl cellulose, glucose/sugar syrup, gelatin, hydroxymethyl cellulose, carboxymethyl cellulose, aluminium stearate gel and/or hydrogenated edible fats), emulsifying agents (e.g. lecithin, sorbitan mono-oleate and/or acacia), non-aqueous vehicles (e.g. edible oils, such as almond oil, fractionated coconut oil, oily esters, propylene glycol and/or ethyl alcohol), and preservatives (e.g. methyl or propyl p-hydroxybenzoates and/or sorbic acid).

The compositions of the present invention have been described in the context of a mouthwash. However, the described embodiments are equally applicable if the composition is formulated in other suitable oral or topical composition dosage forms such as in the form of an oral spray, a gum, an electuary, a gel, a dental floss or tape, or a paste.

For example, the composition may be in the form of a toothpaste, an orally acceptable gel, an orally acceptable gum, or an orally acceptable spray. The composition may also be a component of a dental floss or tape.

Along with the excipients disclosed herein for preparing the oral composition of the invention in the form of a mouthwash, the skilled person would be aware of suitable excipients for formulating the oral composition of the present invention into dosage forms such as a toothpaste, gel, spray, electuary, dental floss or tape and/or gum. These excipients are known in the art.

Biological Tests

Test procedures that may be employed to determine the biological (e.g. bactericidal or antimicrobial) activity of the active ingredients include those known to persons skilled in the art for determining:
(a) bactericidal activity against clinically latent bacteria; and
(b) antimicrobial activity against log phase bacteria.

In relation to (a) above, methods for determining activity against clinically latent bacteria include a determination, under conditions known to those skilled in the art (such as those described in Nature Reviews, Drug Discovery 1, 895-910 (2002), the disclosures of which are hereby incorporated by reference), of Minimum Stationary-cidal Concentration (“MSC”) or Minimum Dormicidal Concentration (“MDC”) for a test compound.

By way of example, WO2000028074 describes a suitable method of screening compounds to determine their ability to kill clinically latent microorganisms. A typical method may include the following steps:
(1) growing a bacterial culture to stationary phase;
(2) treating the stationary phase culture with one or more antimicrobial agents at a concentration and or time sufficient to kill growing bacteria, thereby selecting a phenotypically resistant sub-population;
(3) incubating a sample of the phenotypically resistant subpopulation with one or more test compounds or agents; and
(4) assessing any antimicrobial effects against the phenotypically resistant subpopulation.

According to this method, the phenotypically resistant sub-population may be seen as representative of clinically latent bacteria which remain metabolically active in vivo and which can result in relapse or onset of disease.

In relation to (b) above, methods for determining activity against log phase bacteria include a determination, under standard conditions (i.e. conditions known to those skilled in the art, such as those described in WO2005014585, the disclosures of which document are hereby incorporated by reference), of Minimum Inhibitory Concentration (MIC) or Minimum Bactericidal Concentration (MBC) for a test compound. Specific examples of such methods are described below.

### EXAMPLES

### Example 1: Efficacy of the Antimicrobial Combination

The antimicrobial activity of 4-methyl-8-phenoxy-1-(2-phenylethyl)-2,3-dihydro-1H-pyrrolo[3,2-c]quinoline hydrochloride (HT61 HCl) in combination with chlorhexidine digluconate against S. aureus was assessed at varying concentrations of both actives. The formulations were prepared as shown below in Table 1.

TABLE 1

| | % w/v (F013) | | | | |
|---|---|---|---|---|---|
| Ingredients | (1) | (2) | (3) | (4) | (5) |
| HT61 HCl | 0.005 | 0.01 | 0.02 | 0.001 | 0.01 |
| Chlorhexidine | 0.05 | 0.05 | 0.05 | 0.001 | 0.001 |
| digluconate | | | | | |
| Cremophor ® | 0.7 | 0.7 | 0.7 | 0.7 | 0.7 |
| RH 40 | | | | | |
| Ethanol | 7 | 7 | 7 | 7 | 7 |
| (96.0%) | | | | | |
| Sorbitol | 6.7 | 6.7 | 6.7 | 6.7 | 6.7 |
| (70%) | | | | | |
| Flavour | 1 | 1 | 1 | 1 | 1 |
| cool mint | | | | | |
| Sucralose | 0.25 | 0.25 | 0.25 | 0.25 | 0.25 |
| Brilliant | 0.002 | 0.002 | 0.002 | 0.002 | 0.002 |
| blue colour | | | | | |
| Water | q.s. | q.s. | q.s. | q.s. | q.s. |
| | to 100 | to 100 | to 100 | to 100 | to 100 |

Bacterial Inhibition Assay

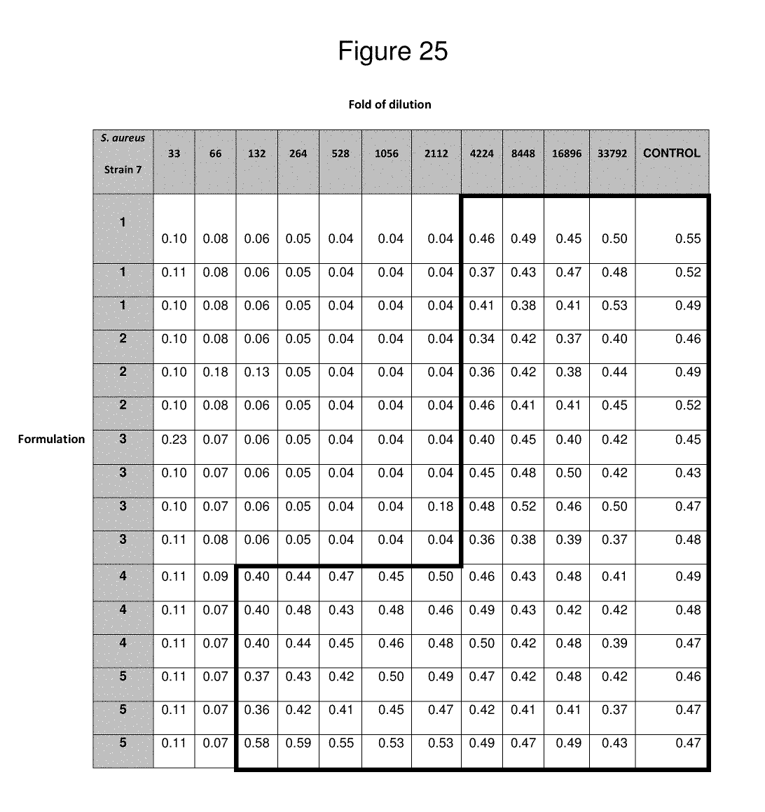

The efficacy of each of the above formulations F013 (1) to (5) was assessed against S. aureus using an inhibition assay. The S. aureus culture was grown according to methods known in the art. The formulations were diluted with the bacterial culture starting at 33 fold to 33792 fold dilution. Optical density of the culture was read after 24 hours of exposure. The results are depicted in FIG. 25

Conclusions:
1. The Minimal Inhibitory Concentrations of formulations (1), (2), and (3) are seen when the formulations were diluted to 2112 fold.
2. The Minimal Inhibitory Concentrations of formulations (4) and (5) are seen when the formulations were diluted to 66 fold.

Time-Kill Studies

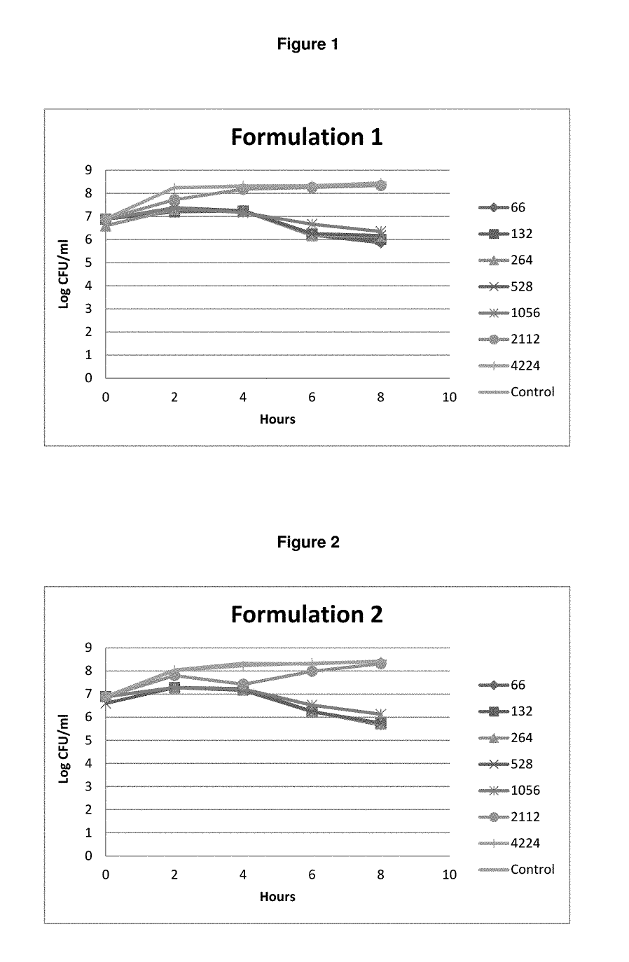

Formulations (1) to (3) were diluted at 66-fold, 132-fold, 264-fold, 528-fold, 1056-fold, 2112-fold and 4224-fold with the S. aureus bacterial culture and CFU counts were performed over 8 hours. FIGS. 1 to 3 contain the time-kill curves for each diluted formulation against log phase S. aureus.

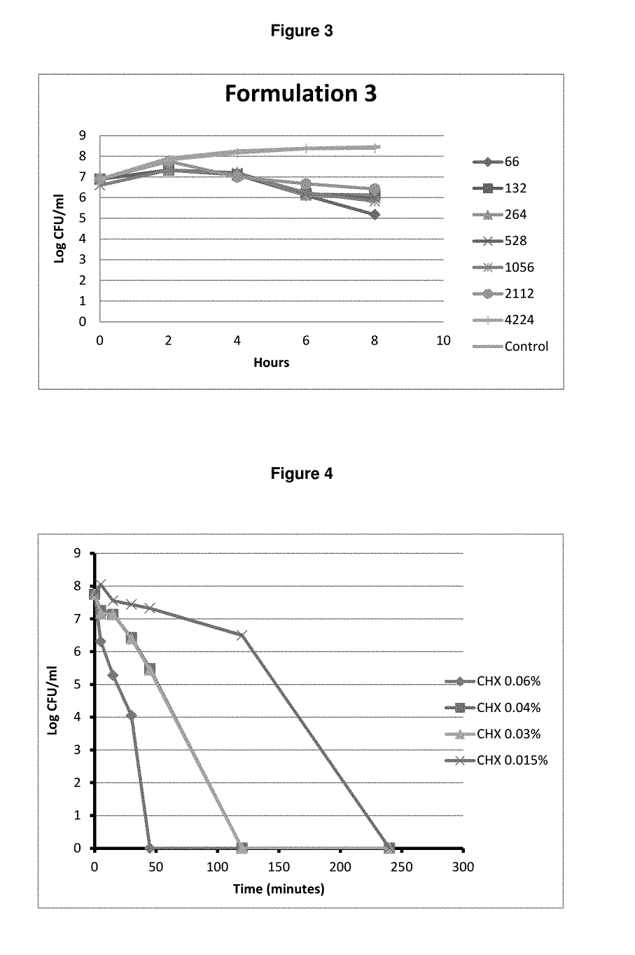

FIG. 1 shows the time-kill curves for the dilutions of formulation (1). FIG. 2 shows the time-kill curves for the dilutions of formulation (2). FIG. 3 shows the time-kill curves for the dilutions of formulation (3).

Each of the three formulations (1) to (3) contained 0.05% w/v chlorhexidine digluconate and either 0.005, 0.01 or 0.02% w/v of HT61 HCl, and it can be seen from FIGS. 1 to 3 that all three formulations inhibited bacterial growth up to 1056 fold dilution at 8 hours.

Formulation (3) can also be seen to provide slightly improved inhibition with bacterial growth inhibited at 2112 fold dilution at 8 hours (FIG. 3).

4-methyl-8-phenoxy-1-(2-phenylethyl)-2,3-dihydro-1H-pyrrolo[3,2-c]quinoline hydrochloride and chlorhexidine digluconate thus exhibit effective antimicrobial activity against log phase S. aureus even with a low concentration of both actives.

### Example 2: Effect of Alcohol on the Efficacy of the Antimicrobial Combination

The effect of alcohol on the antimicrobial activity of HT61 HCl in combination with chlorhexidine digluconate against S. aureus was assessed. The formulations were prepared as shown below in Table 2.

TABLE 2

% w/v (F013)

| Ingredients | (6) | (7) |
|---|---|---|
| HT61 HCl | 0.01 | 0.01 |
| Chlorhexidine | 0.06 | 0.06 |
| digluconate | | |
| Cremophor ® | 0.7 | 0.7 |
| RH 40 | | |
| Ethanol | 7 | 0 |
| (96.0%) | | |
| Sorbitol | 6.7 | 6.7 |
| (70%) | | |
| Flavour | 1 | 1 |
| cool mint | | |
| Sucralose | 0.25 | 0.25 |
| Brilliant | 0.002 | 0.002 |
| blue colour | | |
| Water | q.s. to 100 | q.s. to 100 |

Bacterial Inhibition Assay

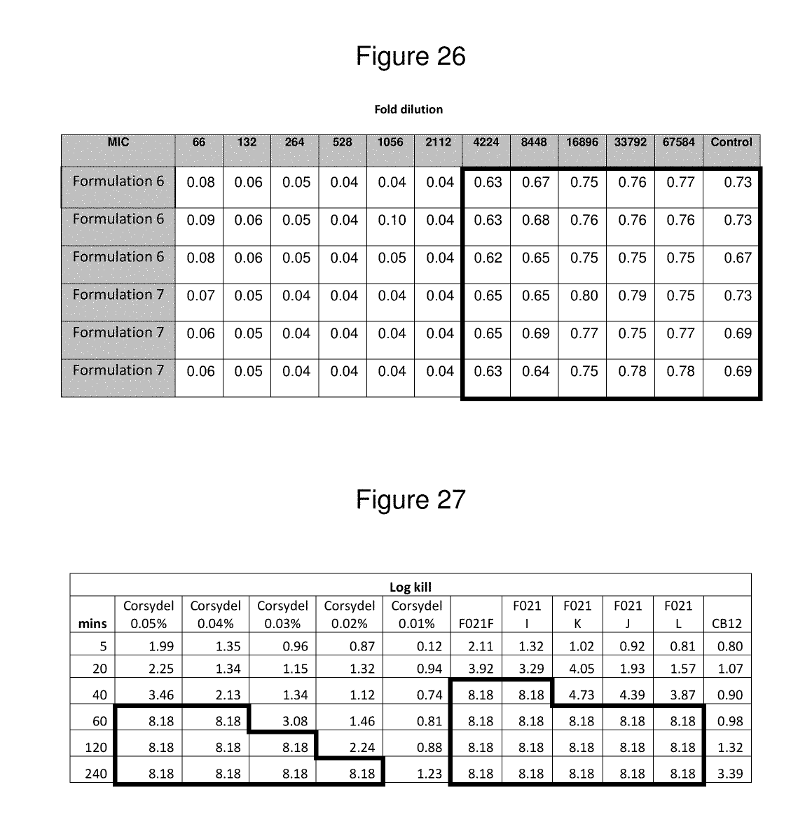

The efficacy of each of the above formulations (6) and (7) was assessed against S. aureus using an inhibition assay. The formulations were diluted with the bacterial culture starting at 66-fold and increasing in stages up to 67584-fold. Optical density of the culture was read after 24 hours of exposure. The results are depicted in FIG. 26

It can be seen that the inclusion of alcohol (here ethanol) has no effect on the minimal inhibitory concentration of the formulation. Both formulations have a MIC when diluted to 2112-fold. This is advantageous because it means that the oral compositions of the present invention with effective antimicrobial activity can be prepared with or without alcohol.

### Example 3: Efficacy of the Antimicrobial Combination

To further investigate the antimicrobial activity of HT61 HCl in combination with chlorhexidine digluconate, experiments were carried out against S. aureus using 0.01% w/v HT61 HCl with varying concentrations of chlorhexidine digluconate.

The formulations were prepared as shown below in Table 3.

TABLE 3

| | F016 A | | | F016 B | | | F016 C | | | F016 D | | | F016 E | | |
|---|---|---|---|---|---|---|---|---|---|---|---|---|---|---|---|
| | | Qty/ | | | Qty/ | | | Qty/ | | | Qty/ | | | Qty/ | |
| Ingredients | % w/v | 100 ml | | % w/v | 100 ml | | % w/v | 100 ml | | % w/v | 100 ml | | % w/v | 100 ml | |
| HT61 HCl | 0.01 | 10.162 | mg | 0.01 | 10.162 | mg | 0.01 | 10.162 | mg | 0.01 | 10.162 | mg | 0.01 | 10.162 | mg |
| Chlorhexidine | 0.06 | 0.310 | g | 0.04 | 0.206 | g | 0.03 | 0.155 | g | 0.015 | 0.077 | g | 0.06 | 0.310 | g |
| digluconate | | | | | | | | | | | | | | | |
| Cremophor ® | 0.7 | 0.7 | g | 0.7 | 0.7 | g | 0.7 | 0.7 | g | 0.7 | 0.7 | g | 0 | 0 | |
| RH 40 | | | | | | | | | | | | | | | |
| Ethanol | 7 | 7.0 | ml | 7 | 7.0 | ml | 7 | 7.0 | ml | 7 | 7.0 | ml | 7 | 7.0 | ml |
| (96.0%)* | | | | | | | | | | | | | | | |
| Sorbitol 70% | 6.7 | 6.7 | g | 6.7 | 6.7 | g | 6.7 | 6.7 | g | 6.7 | 6.7 | g | 6.7 | 6.7 | g |
| Flavour cool | 1 | 1.0 | g | 1 | 1.0 | g | 1 | 1.0 | g | 1 | 1.0 | g | 1 | 1.0 | g |
| mint | | | | | | | | | | | | | | | |
| Sucralose | 0.25 | 0.25 | g | 0.25 | 0.25 | g | 0.25 | 0.25 | g | 0.25 | 0.25 | g | 0.25 | 0.25 | g |
| Brilliant blue | 0.002 | 2.0 | mg | 0.002 | 2.0 | mg | 0.002 | 2.0 | mg | 0.002 | 2.0 | mg | 0.002 | 2.0 | mg |
| colour | | | | | | | | | | | | | | | |

| | | | | | |
|---|---|---|---|---|---|
| Purified Water | q.s. to 100 ml | q.s. to 100 ml | q.s. to 100 ml | q.s. to 100 ml | q.s. to 100 ml |
| Physical | Clear solution | Clear solution | Clear solution | Clear solution | Translucent |
| observation | | | | | solution |

Time-Kill Studies

FIGS. 4, 5 and 6 contain the results of the time-kill studies then carried out against log phase S. aureus for 240 minutes (4 hours).

FIG. 4 shows the time-kill curves for the chlorhexidine formulations CHX 0.06%, CHX 0.04%, CHX 0.03% and CHX 0.015%. These formulations included chlorhexidine digluconate at either 0.06% w/v, 0.04% w/v, 0.03% w/v or 0.015% w/v.

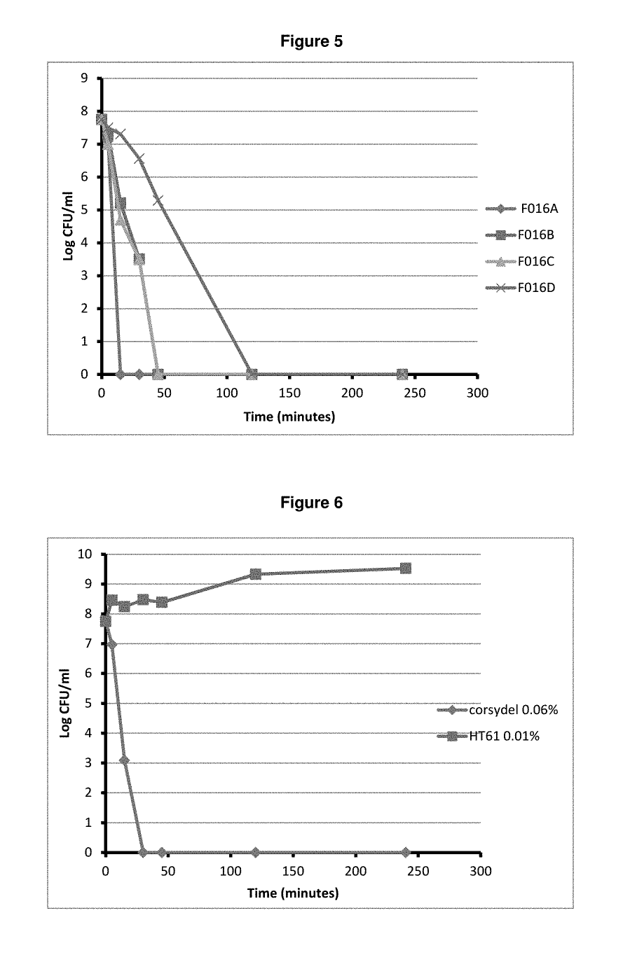

FIG. 5 shows the time-kill curves for the formulations F016A, F016B, F016C and F016D.

FIG. 6 shows the time-kill curves for Corsodyl® Daily Defence mouthwash 0.06% and a formulation solely containing 0.01% w/v HT61 HCl. Corsodyl® Daily Defence mouthwash 0.06% is a commercially available product manufactured by GlaxoSmithKline which contains 0.06% w/v chlorhexidine digluconate.

The time to zero values for each of the FIGS. 4 to 6 are shown below in Table 4.

TABLE 4

| | CHX 0.06% | CHX 0.04% | CHX 0.03% | CHX 0.015% |
|---|---|---|---|---|
| Time to zero | 45 | 120 | 120 | 240 |
| (minutes) | | | | |

| | F016A | F016B | F016C | F016D |
|---|---|---|---|---|
| Time to zero | 15 | 45 | 45 | 120 |
| (minutes) | | | | |

| | Corsodyl ® 0.06% |
|---|---|
| Time to zero | 30 |
| (minutes) | |
| | HT61 0.01% |
| Time to zero | inactive |
| (minutes) | |

It can be seen from these time to zero values (and from comparing FIGS. 4 and 5) that HT61 HCl and chlorhexidine digluconate have effective antimicrobial activity against log phase S. aureus even at low concentrations of chlorhexidine. The addition of 0.01% w/v HT61 HCl to the formulation significantly improves the time to zero value for each concentration of chlorhexidine digluconate:
At 0.06% w/v chlorhexidine digluconate, the time to zero reduces from 45 to 15 minutes.
At 0.04% w/v and 0.03% w/v chlorhexidine digluconate the time to zero reduces from 120 to 45 minutes.
At 0.015% w/v chlorhexidine digluconate the time to zero reduces from 240 to 120 minutes.

FIG. 6 also shows that HT61 alone at 0.01% w/v does not have significant antimicrobial activity against log phase S. aureus. The antimicrobial activity seen for the compositions of the present invention is therefore arising from the combination of actives.

It can also be seen from these time to zero values that the composition of the present invention has improved antimicrobial activity against S. aureus compared to Corsodyl® Daily Defence Mouthwash. That's to say, with the same concentration of chlorhexidine (0.06% w/v), the inclusion of HT61 HCl improves the time to zero value from 30 to 15 minutes (F016A). The composition of the present invention therefore demonstrates more effective antimicrobial activity compared with the commercially available product.

Furthermore, even with a lower amount of chlorhexidine (0.04% and 0.03% w/v in F016B and F016C), the composition of the present invention still provides antimicrobial activity with a time to zero value of 45 minutes.

These results demonstrate that the oral composition of the invention including about 0.001% w/v to about 0.06% w/v of chlorhexidine or a pharmaceutically acceptable salt and/or solvate thereof in combination with HT61 or a pharmaceutically acceptable salt and/or solvate thereof provides an improved mouthwash over the commercially available 0.06% w/v chlorhexidine mouthwash product.

### Example 4: Effect of Surfactant on Antimicrobial Activity

The effect of surfactant on the antimicrobial activity of HT61 HCl in combination with chlorhexidine digluconate against S. aureus was also assessed. The formulations without surfactant (here Cremophor® RH 40) were prepared as shown below in Table 5. These were then compared against formulations with surfactant as prepared in the above Examples.

TABLE 5

| | F021 | | | | | | | |
|---|---|---|---|---|---|---|---|---|
| Ingredients | | | | | | | | Pla- |
| (qty % w/v) | F | I | J | K | L | M | N | cebo |
| HT61 HCl | 0.01 | 0.01 | 0.01 | 0.01 | 0.01 | 0 | 0.01 | 0 |
| Chlorhexidine | 0.05 | 0.04 | 0.03 | 0.02 | 0.01 | 0.05 | 0 | 0 |
| digluconate | | | | | | | | |
| Cremophor ® | 0 | 0 | 0 | 0 | 0 | 0 | 0 | 0 |
| RH 40 | | | | | | | | |
| Ethanol | 7 | 7 | 7 | 7 | 7 | 7 | 7 | 7 |
| (96.0%)v/v | | | | | | | | |
| Sorbitol 70% | 6.7 | 6.7 | 6.7 | 6.7 | 6.7 | 6.7 | 6.7 | 6.7 |
| Flavour - Old | 0 | 0 | 0 | 0 | 0 | 0 | 0 | 0 |
| Flavour - New | 0.5 | 0.5 | 0.5 | 0.5 | 0.5 | 0.5 | 0.5 | 0.5 |
| (High water- | | | | | | | | |
| solubility) | | | | | | | | |
| Sucralose | 0.25 | 0.25 | 0.25 | 0.25 | 0.25 | 0.25 | 0.25 | 0.25 |

Time-Kill Studies

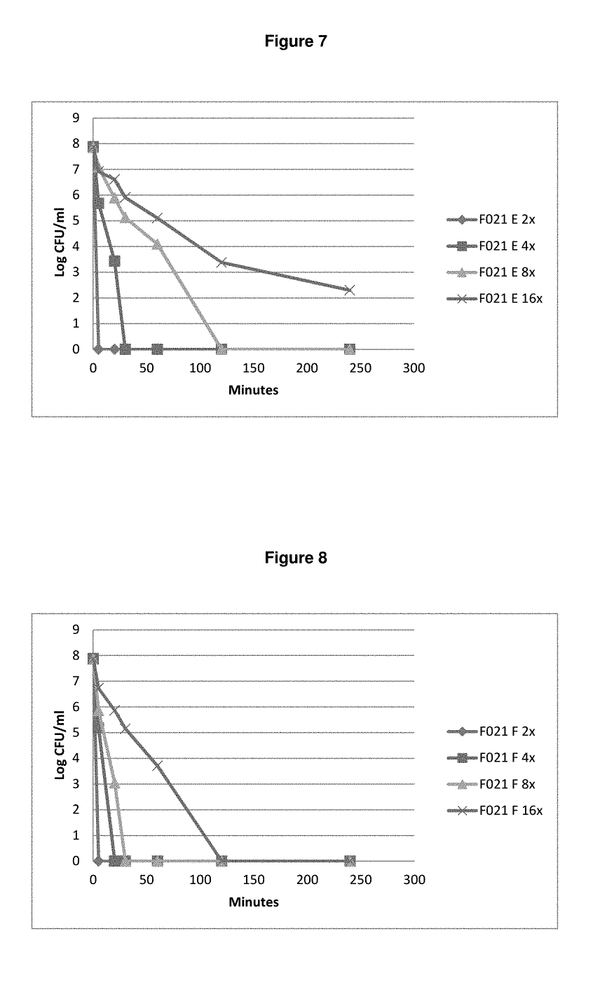

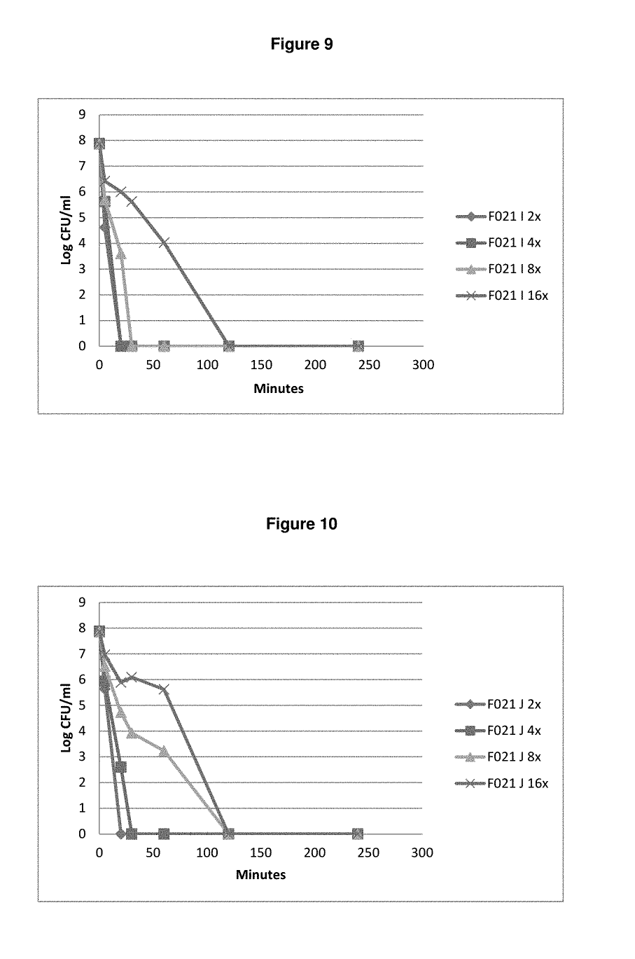

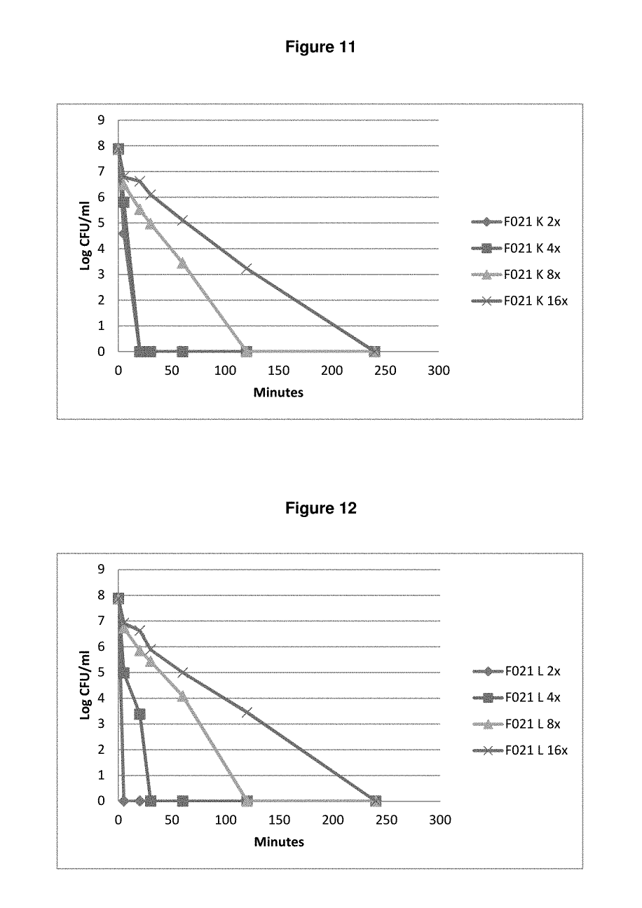

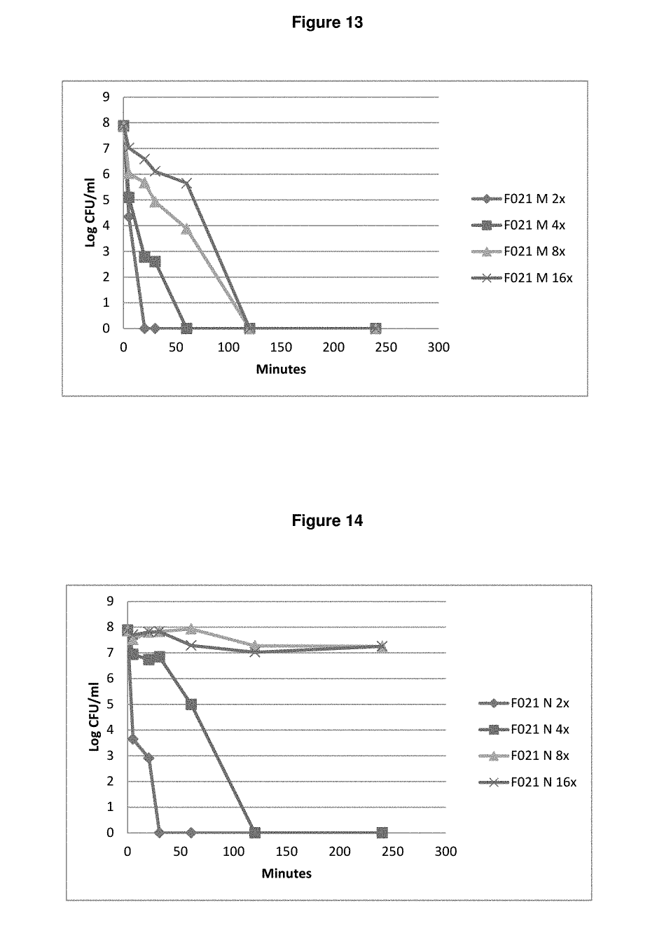

FIGS. 7 to 14 contain the results of time-kill studies over 240 minutes against log phase S. aureus.

FIG. 7 shows the time-kill curve for the F021E formulation at 2-fold, 4-fold, 8-fold and 16-fold dilution. The F021E formulation is set out above in Table 3: it contains 0.01% w/v HT61 HCl, 0.06% w/v chlorhexidine gluconate and no surfactant (Cremophor® RH 40)

FIGS. 8 to 14 then shows the time-kill curves for the F021F, F021I, F021J, F021K, F021L, F021M and F021N formulations at 2-fold, 4-fold, 8-fold and 16-fold dilution.

The time to zero values from the time-kill studies shown in FIGS. 7 to 14 are set out below in Tables 6 to 12.

TABLE 6

| Minutes | F021 E 2x | F021 E 4x | F021 E 8x | F021 E 16x |
|---|---|---|---|---|
| 5 | 7.88 | 2.20 | 0.79 | 0.97 |
| 20 | 7.88 | 4.45 | 1.99 | 1.27 |
| 30 | 7.88 | 7.88 | 2.76 | 1.97 |
| 60 | 7.88 | 7.88 | 3.79 | 2.78 |
| 120 | 7.88 | 7.88 | 7.88 | 4.50 |
| 240 | 7.88 | 7.88 | 7.88 | 5.58 |
| Time to zero | 5 | 30 | 120 | NA |

TABLE 7

| Minutes | F021 F 2x | F021 F 4x | F021 F 8x | F021 F 16x |
|---|---|---|---|---|
| 5 | 7.88 | 2.70 | 2.02 | 1.16 |
| 20 | 7.88 | 7.88 | 4.84 | 2.02 |
| 30 | 7.88 | 7.88 | 7.88 | 2.73 |
| 60 | 7.88 | 7.88 | 7.88 | 4.17 |
| 120 | 7.88 | 7.88 | 7.88 | 7.88 |
| 240 | 7.88 | 7.88 | 7.88 | 7.88 |
| Time to zero | 5 | 20 | 30 | 120 |

TABLE 8

| Minutes | F021 I 2x | F021 I 4x | F021 I 8x | F021 I 16x |
|---|---|---|---|---|
| 5 | 3.27 | 2.26 | 2.18 | 1.47 |
| 20 | 7.88 | 7.88 | 4.28 | 1.88 |
| 30 | 7.88 | 7.88 | 7.88 | 2.26 |
| 60 | 7.88 | 7.88 | 7.88 | 3.86 |
| 120 | 7.88 | 7.88 | 7.88 | 7.88 |
| 240 | 7.88 | 7.88 | 7.88 | 7.88 |
| Time to zero | 20 | 20 | 30 | 120 |

TABLE 9

| Minutes | F021 J 2x | F021 J 4x | F021 J 8x | F021 J 16x |
|---|---|---|---|---|
| 5 | 2.25 | 1.94 | 1.36 | 0.92 |
| 20 | 7.88 | 5.28 | 3.16 | 1.99 |
| 30 | 7.88 | 7.88 | 3.96 | 1.79 |
| 60 | 7.88 | 7.88 | 4.65 | 2.26 |
| 120 | 7.88 | 7.88 | 7.88 | 7.88 |
| 240 | 7.88 | 7.88 | 7.88 | 7.88 |
| Time to zero | 20 | 30 | 120 | 120 |

TABLE 10

| Minutes | F021 L 2x | F021 L 4x | F021 L 8x | F021 L 16x |
|---|---|---|---|---|
| 5 | 7.88 | 2.90 | 1.15 | 0.96 |
| 20 | 7.88 | 4.50 | 2.03 | 1.26 |
| 30 | 7.88 | 7.88 | 2.45 | 1.99 |
| 60 | 7.88 | 7.88 | 3.80 | 2.89 |
| 120 | 7.88 | 7.88 | 7.88 | 4.43 |
| 240 | 7.88 | 7.88 | 7.88 | 7.88 |
| Time to zero | 5 | 30 | 120 | 240 |

TABLE 11

| Minutes | F021 M 2x | F021 M 4x | F021 M 8x | F021 M 16x |
|---|---|---|---|---|
| 5 | 3.54 | 2.78 | 1.85 | 0.86 |
| 20 | 7.88 | 5.10 | 2.21 | 1.29 |
| 30 | 7.88 | 5.28 | 2.95 | 1.77 |
| 60 | 7.88 | 7.88 | 4.00 | 2.23 |
| 120 | 7.88 | 7.88 | 7.88 | 7.88 |
| 240 | 7.88 | 7.88 | 7.88 | 7.88 |
| Time to zero | 20 | 60 | 120 | 120 |

TABLE 12

| Minutes | F021 N 2x | F021 N 4x | F021 N 8x | F021 N 16x |
|---|---|---|---|---|
| 5 | 4.24 | 0.94 | 0.36 | 0.18 |
| 20 | 4.98 | 1.15 | 0.08 | 0.07 |
| 30 | 7.88 | 1.03 | 0.05 | 0.05 |
| 60 | 7.88 | 2.89 | −0.05 | 0.59 |
| 120 | 7.88 | 7.88 | 0.60 | 0.85 |
| 240 | 7.88 | 7.88 | 0.63 | 0.63 |
| Time to zero | 30 | 120 | NA | NA |

It can be seen by comparing the time to zero for F016E and F016A (see Table 4) that the removal of surfactant improves the antimicrobial activity of the composition. At 2-fold dilution the time to zero decreased from 15 minutes (F016A) to 5 minutes (F016E).

The same trend can be seen by comparing the time to zero for F016B (see Table 4: 45 minutes) and F021I at two-fold dilution (20 minutes); and F021J with F016C (20 minutes vs. 45 minutes). The absence of a surfactant results in an improved time to zero and hence improved antimicrobial activity.

Additionally from comparing the time to zero for F021F and F021M, it can be seen that the addition of 0.01% HT61 HCl significantly improves the antimicrobial activity of the combination. The time to zero decreases from 20 minutes to 5 minutes at 2-fold dilution, 60 minutes to 20 minutes at 4-fold dilution and 120 minutes to 30 minutes at 8-fold dilution. Furthermore, F021L contains 0.01% w/v HT61 HCl and 0.01% w/v chlorhexidine digluconate without surfactant and has a surprising time to zero value of only 5 minutes at 2-fold dilution.

It is therefore advantageous for the oral composition to not include a surfactant.

Mouthwash compositions F01 (with surfactant) and F02 (without surfactant) were also prepared for assessment of antimicrobial activity. These compositions are disclosed in Table 13 and their antimicrobial activity against S. aureus is reported in Table 4. The antimicrobial activity was measured by evaluating the time required to achieve zero log CFU per ml.

TABLE 13

Batch

| | F01 | F02 |
|---|---|---|
| Ingredients | % w/v | % w/v |
| Chlorhexidine | 0.06 | 0.06 |
| HT61 HCl | 0.01 | 0.01 |
| Cremophor ® | 0.7 | 0.0 |
| RH40 | | |
| Ethanol | 7.0 | 7.0 |
| Sorbitol | 6.7 | 6.7 |
| Flavour | 0.5 | 0.5 |
| Sucralose | 0.25 | 0.25 |
| Colour | 0.002 | 0.002 |
| Water (q.s. to | q.s | q.s |
| 100 ml) | | |

TABLE 14

Time required (in minutes) to attain
0 log CFU/ml for formulation

| Dilutions | F01 | F02 |
|---|---|---|
| 8 x | 420 | 50 |
| 16 x | Not attained | 240 |

It can be seen from Table 14 that the time required to attain zero log CFU/ml increased when surfactant was used in the formulation. These results also therefore show that it is advantageous not to include a surfactant in the composition of the invention.

### Example 5: Comparison of F021 Formulations with Corsodyl® Daily and CB12

To demonstrate the improved efficacy of the oral compositions of the invention over commercially available products containing low concentrations of chlorhexidine, a comparison was made between formulations F021F to L and Corsodyl® Daily diluted to the comparable % w/v of chlorhexidine using time-kill studies. A comparison was also made between the time-kill curves obtained for F021L (containing 0.01% w/v chlorhexidine digluconate and 0.01% w/v HT61 HCl) and CB12.

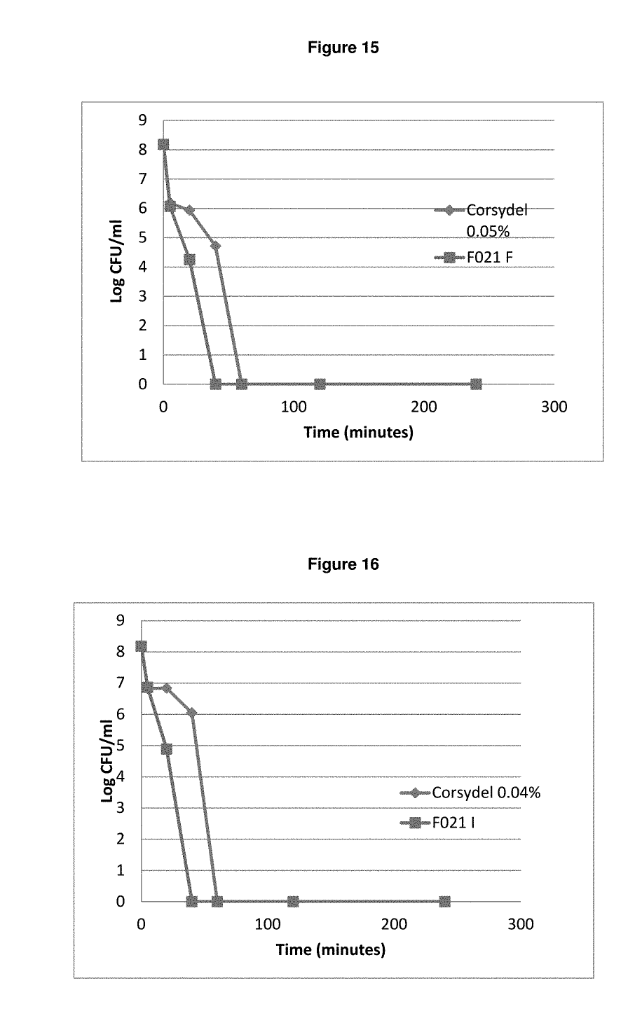

These comparisons are shown in FIGS. 15 to 20.

FIG. 15 shows the time-kill curves for the F021F formulation and for Corsodyl® Daily diluted to a chlorhexidine concentration of 0.05% w/v.

FIG. 16 shows the time-kill curves for the F021I formulation and for Corsodyl® Daily diluted to a chlorhexidine concentration of 0.04% w/v.

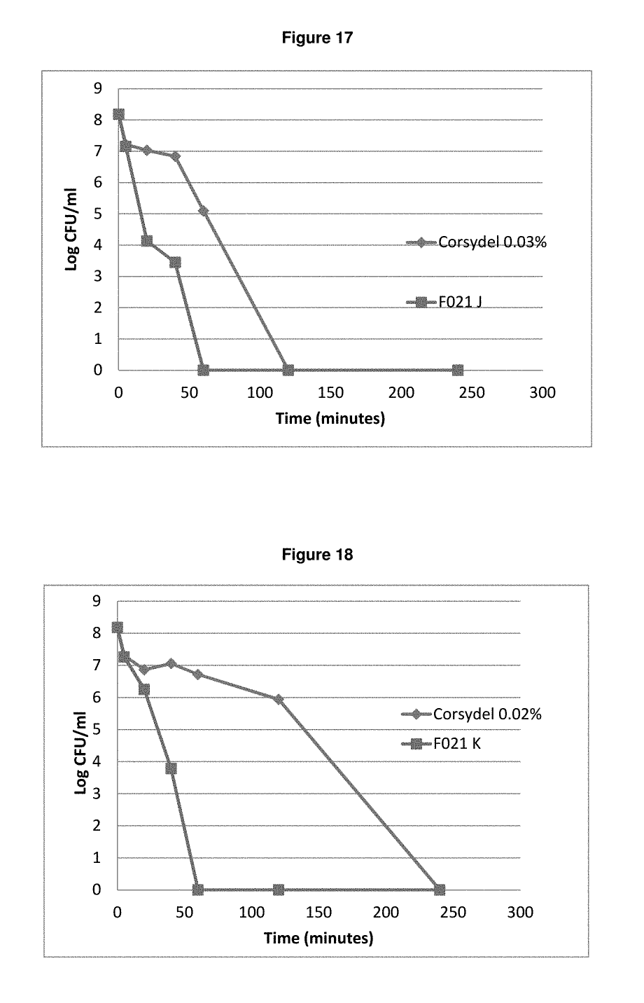

FIG. 17 shows the time-kill curves for the F021J formulation and for Corsodyl® Daily diluted to a chlorhexidine concentration of 0.03% w/v.

FIG. 18 shows the time-kill curves for the F021K formulation and for Corsodyl® Daily diluted to a chlorhexidine concentration of 0.02% w/v.

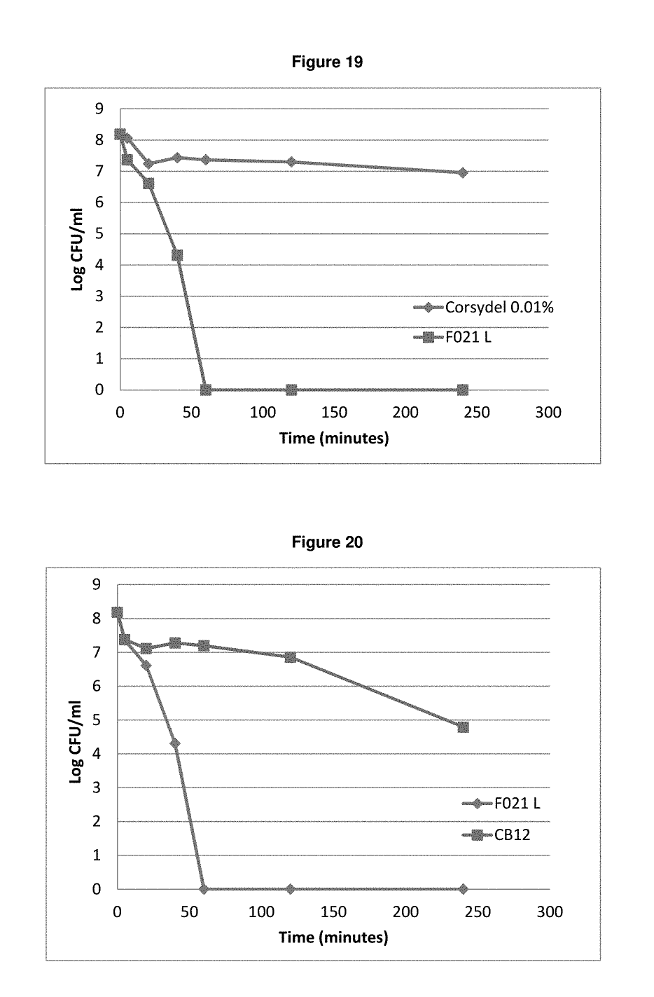

FIG. 19 shows the time-kill curves for the F021L formulation and for Corsodyl® Daily diluted to a chlorhexidine concentration of 0.01% w/v.

FIG. 20 shows the time-kill curves for the F021L formulation and for CB12.

It can be seen from FIGS. 15 to 19 that each of the formulations F021F to L has an improved time to zero over the comparable Corsodyl® Daily formulation. The most significant improvement can be seen in FIG. 19 with the F021 L formulation compared with Corsodyl® Daily diluted to 0.01%.

It can further be seen from FIG. 20 that the formulation F021 L has a much faster time to zero than commercially available CB12.

The improvements are also identifiable from FIG. 27, where complete kill is shown by the shaded cells. The CFU count at time zero was log 8.18 so a log kill of 8.18 indicates 100% kill.

The oral composition of the present invention thus exhibits improved antibacterial activity (shown by a faster time to zero) over commercially available Corsodyl® Daily at a comparable chlorhexidine digluconate concentration. The presence of HT61 in the composition means that chlorhexidine is still effective even at a low concentration.

Further comparative experiments have been carried out against Corsodyl® Daily Mouthwash (0.06% w/v chlorhexidine). Mouthwash compositions containing varying concentrations of chlorhexidine (0.02, 0.01 or 0.00% w/v) and 0.01% w/v HT61 were prepared and their antimicrobial activity against S. aureus compared with that of Corsodyl® Daily. The compositions are disclosed in Table 15 and the antimicrobial activity evaluated in Table 16.

TABLE 15

| | Batch | | |
|---|---|---|---|
| | F03 | F04 | F05 |
| Ingredients | % w/v | % w/v | % w/v |
| Chlorhexidine | 0.02 | 0.01 | — |
| HT61 HCl | 0.01 | 0.01 | 0.01 |
| Ethanol | 7.0 | 7.0 | 7.0 |
| Sorbitol | 6.7 | 6.7 | 6.7 |
| Flavour | 0.5 | 0.5 | 0.5 |
| Sucralose | 0.25 | 0.25 | 0.25 |
| Colour | 0.002 | 0.002 | 0.002 |
| Water (q.s. to | q.s. | q.s. | q.s. |
| 100 ml) | | | |

TABLE 16

| | Time required (in minutes) to attain | | | |
|---|---|---|---|---|
| | 0 log CFU/ml for formulation | | | |
| Dilutions | Corsodyl ® Daily Mouthwash | F03 | F04 | F05 |
| 8 x | 120 | 120 | 120 | Not attained |
| 16 x | 240 | 240 | 240 | Not attained |

It can be seen from the results in Table 16 that the mouthwash compositions of the present invention with a low amount of chlorhexidine (0.02, 0.01% w/v), are effective against S. aureus. It can also be seen that the mouthwash composition containing 0.025% w/v or less chlorhexidine and 0.01% w/v HT61 provides antimicrobial activity equivalent to 0.06% w/v of chlorhexidine (Corsodyl® Daily Mouthwash).

### Example 6: Oral Compositions Including a Zinc Compound

The antimicrobial activity of 4-methyl-8-phenoxy-1-(2-phenylethyl)-2,3-dihydro-1H-pyrrolo[3,2-c]quinoline acetate (HT61 acetate) in combination with chlorhexidine diacetate (CHD diacetate) and zinc acetate against S. aureus was assessed. The formulations were prepared as shown below in Table 17.

TABLE 17

| Ingredients | F029 | | |
|---|---|---|---|
| (qty % w/v) | A | B | C |
| CHD Diacetate | 0.01% | 0.03% | 0.03% |
| HT61 Acetate | 0.01% | 0.03% | 0.03% |
| Zinc Acetate | 0.07% | 0.07% | 0.30% |
| Sodium. Fluoride | 0.05% | 0.05% | 0.05% |
| Sorbitol 70% | 6.7 | 6.7 | 6.7 |
| Flavour | 0.5 | 0.5 | 0.5 |
| Sucralose | 0.25 | 0.25 | 0.25 |
| Water | q.s to 100 | q.s to 100 | q.s to 100 |
| Conc. HCl | 0 | 15 μl | 25 μl |
| Observation | Clear solution | Clear solution | Clear solution |
| pH | pH 6.3-6.5 | pH 5.5-5.75 | pH 5.25-5.5 |

Time-Kill Curves

The formulations F029A, F029B and F029C together with the commercially available CB12 (containing 0.3% w/v zinc acetate and 0.025% w/v chlorhexidine diacetate) were each diluted at 2-fold with a culture of the bacterium S. aureus. CFU counts were then performed at 0, 5, 10, 15, 20, 30, 40, 50, 60 and 120 minutes after exposure.

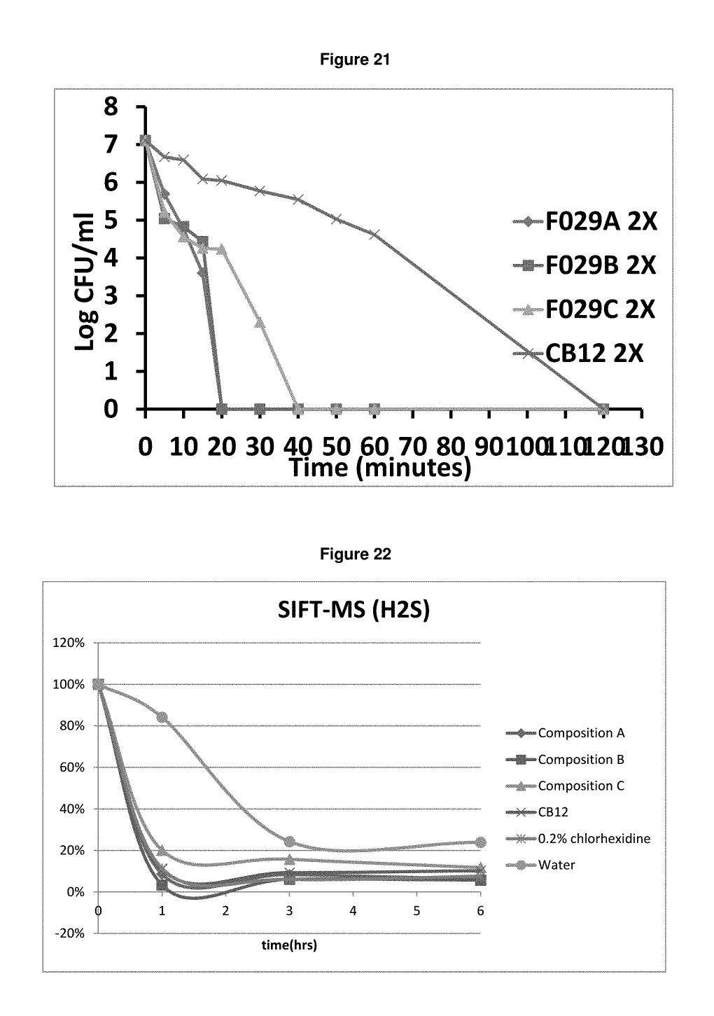

FIG. 21 contains the time-kill curves for each of the tested formulations against log phase S. aureus.

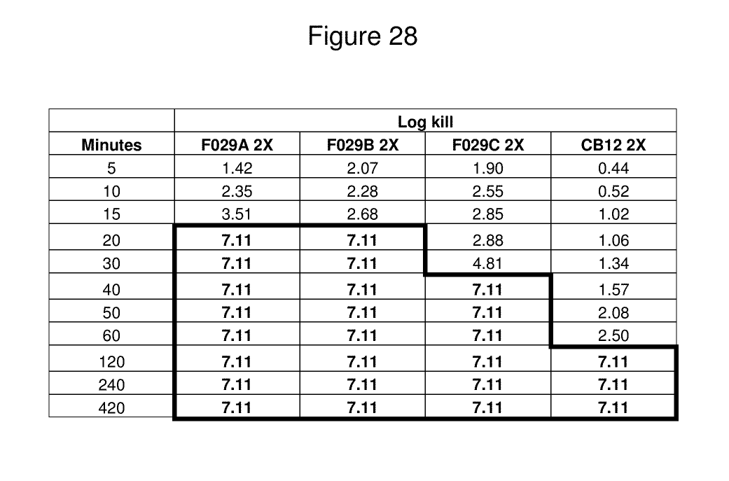

It can be seen from FIG. 21 that all of the compositions of the invention F029A to C had a faster time to zero tan the commercially available CB12. The specific CFU values are set out in FIG. 28.

The CFU count at time zero was log 7.11 so a log kill of 7.11 indicates 100% kill. It can thus be seen from FIG. 28 that the time needed for F029A and F029B to achieve 100% kill was 20 minutes, and that the time needed for F029C to achieve 100% kill was 30 minutes. In contrast, the time needed for CB12 to achieve 100% kill was 120 minutes.

The oral composition of the present invention thus exhibits improved antibacterial activity (shown by a faster time to zero value) over the commercially available CB12 and the combination of HT61, chlorhexidine and a zinc compound such as zinc acetate demonstrates a synergistic effect against log phase S. aureus.

### Example 7: In Vivo Data

Example 7 evaluates the effect of oral compositions of the present invention on oral malodour after 1 hour, 3 hours and 6 hours following use of the composition.

Oral malodour may arise from organisms on the surface of the tongue. In particular, it is thought that certain Gram-negative anaerobes are responsible for this condition. They possess enzymes that allow biotransformation of sulphur-substrates (cysteine, methionine and glutathione) into volatile sulphur compounds. By testing formulations (three test solutions A to C with active ingredients) against one negative control (water), and two positive controls (CB12 and chlorhexidine di-gluconate 0.2% w/v) information may be gained as to the efficacy of test compounds in terms of their immediate (within 30-60 minutes), intermediate (3 hours) and longer (6 hours) effects on oral malodour. The formulations were tested by measuring breath parameters using selected ion flow tube mass spectrometry and by sampling of tongue biofilm for numbers of viable microbial species.

Subject Selection:

Eligibility Criteria

1. Voluntary participation in the study as documented on a subject informed consent form.

2. Availability at the investigational site at the specified study intervals and testing times.

3. Organoleptic score>2 and H₂S>100 ppb

Exclusion Criteria

1. Medical history of infectious diseases (e.g. hepatitis, HIV, tuberculosis)

2. Rampant caries, severe gingivitis, advanced periodontitis, oral thrush.

3. Antibiotic medication within 1 month prior to the start of the trial or during the trial period.

4. Consumption of medicated sweets containing antimicrobial agents.

5. Changes in oral hygiene practices during the trial

6. Consumption of foods associated with oral malodour (e.g. garlic) on the day prior to, and on the testing day, and wearing of strongly perfumed cosmetics on the testing day.

7. Substantial false dentition.

Study

The 32 volunteers (subjects) who were eligible to enter the trial were randomised and attended the laboratory once a week for six weeks. All subjects used all treatments, i.e. water (F), 0.2% w/v chlorhexidine (E), commercially available CB12 mouthwash (D) or one of the oral compositions of the claimed invention A, B or C detailed below in Table 18. CB12 mouthwash (manufactured by Meda Pharmaceuticals, UK) includes 0.025% w/v chlorhexidine diacetate along with 0.3% w/v zinc acetate.

TABLE 18

| | Composition A | Composition B | Composition C | |
|---|---|---|---|---|
| Component | (% w/v) | (% w/v) | (% w/v) | CB12 |
| Chlorhexidine | 0.025 | 0.025 | 0.01 | 0.025 |
| HT61 | 0.03 | 0.03 | 0.01 | 0 |
| Zinc Acetate | 0.07 | 0.3 | 0.07 | 0.3 |

Each subject was randomly assigned the label 1 to 32. Each subject was ultimately his or her own control. On the first visit, 32 subjects were allocated one treatment A-F. However, only 30 subjects received one of the products as two failed the eligibility criteria on testing day and were withdrawn or excluded. On all other visits over the six week period, 30 subjects were allocated and received one of the treatments A-F. Finally, 30 subjects completed the study and the analysis of the data was based on n=30 since no further withdrawals occurred.

Table 19 below is a summary table showing all enrolled subjects (n=32), their treatment allocation (A, B, C, D, E and F), their fulfillment of exclusion criteria on testing day (Y=Yes, N=No) the number of subjects excluded (n=2), the number of dropouts (n=0), the number of subjects who completed the study (n=30) and the number of subjects who presented any adverse reaction (n=0) and for subjects excluded from the study (NA=not applicable).

TABLE 19

| | | Exclusion | | Completeness | Adverse |
|---|---|---|---|---|---|
| Subject | Allocation | criteria | Dropout | data | Reaction |
| 1 | 1. A | Y | N | Y | N |
| | 2. E | | | | |
| | 3. D | | | | |
| | 4. C | | | | |
| | 5. B | | | | |
| | 6. F | | | | |
| 2 | 1. F | Y | N | Y | N |
| | 2. B | | | | |
| | 3. A | | | | |
| | 4. E | | | | |
| | 5. D | | | | |
| | 6. C | | | | |
| 3 | 1. B | Y | N | Y | N |
| | 2. F | | | | |
| | 3. D | | | | |
| | 4. E | | | | |
| | 5. A | | | | |
| | 6. C | | | | |
| 4 | 1. E | Y | N | Y | N |
| | 2. C | | | | |
| | 3. F | | | | |
| | 4. B | | | | |
| | 5. A | | | | |
| | 6. D | | | | |
| 5 | 1. A | Y | N | Y | N |
| | 2. E | | | | |
| | 3. B | | | | |
| | 4. F | | | | |
| | 5. D | | | | |
| | 6. C | | | | |
| 6 | 1. A | Y | N | Y | N |
| | 2. D | | | | |
| | 3. C | | | | |
| | 4. B | | | | |
| | 5. F | | | | |
| | 6. E | | | | |
| 7 | 1. C | Y | N | Y | N |
| | 2. B | | | | |
| | 3. F | | | | |
| | 4. A | | | | |
| | 5. E | | | | |
| | 6. D | | | | |
| 8 | 1. D | Y | N | Y | N |
| | 2. F | | | | |
| | 3. A | | | | |
| | 4. E | | | | |
| | 5. B | | | | |
| | 6. C | | | | |
| 9 | 1. E | Y | N | Y | N |
| | 2. F | | | | |
| | 3. D | | | | |
| | 4. A | | | | |
| | 5. B | | | | |
| | 6. C | | | | |
| 10 | 1. A | Y | N | Y | N |
| | 2. B | | | | |
| | 3. E | | | | |
| | 4. D | | | | |
| | 5. C | | | | |
| | 6. F | | | | |
| 11 | 1. B | Y | N | Y | N |
| | 2. A | | | | |
| | 3. C | | | | |
| | 4. D | | | | |
| | 5. F | | | | |
| | 6. E | | | | |
| 12 | 1. C | Y | N | Y | N |
| | 2. E | | | | |
| | 3. F | | | | |
| | 4. A | | | | |
| | 5. B | | | | |
| | 6. D | | | | |
| 13 | 1. E | Y | N | Y | N |
| | 2. B | | | | |
| | 3. F | | | | |
| | 4. C | | | | |
| | 5. A | | | | |
| | 6. D | | | | |
| 14 | 1. A | Y | N | Y | N |
| | 2. B | | | | |
| | 3. C | | | | |
| | 4. D | | | | |
| | 5. E | | | | |
| | 6. F | | | | |
| 15 | 1. B, | Y | N | Y | N |
| | 2. F, | | | | |
| | 3. D | | | | |
| | 4. A | | | | |
| | 5. E | | | | |
| | 6. C | | | | |
| 16 | 1. F | Y | N | Y | N |
| | 2. D | | | | |
| | 3. B | | | | |
| | 4. E | | | | |
| | 5. C | | | | |
| | 6. A | | | | |
| 17 | 1. C | Y | N | Y | N |
| | 2. F | | | | |
| | 3. A | | | | |
| | 4. B | | | | |
| | 5. D | | | | |
| | 6. E | | | | |
| 18 | 1. E | Y | N | Y | N |
| | 2. D | | | | |
| | 3. C | | | | |
| | 4. A | | | | |
| | 5. F | | | | |
| | 6. B | | | | |
| 19 | 1. A | Y | N | Y | N |
| | 2. B | | | | |
| | 3. F | | | | |
| | 4. D | | | | |
| | 5. C | | | | |
| | 6. E | | | | |
| 20 | 1. B | Y | N | N | N |
| | 2. A | | | | |
| | 3. E | | | | |
| | 4. D | | | | |
| | 5. C | | | | |
| | 6. F | | | | |
| 21 | 1. A | Y | N | Y | N |
| | 2. B | | | | |
| | 3. D | | | | |
| | 4. E | | | | |
| | 5. F | | | | |
| | 6. C | | | | |
| 22 | 1. D | Y | N | Y | N |
| | 2. B | | | | |
| | 3. A | | | | |
| | 4. F | | | | |
| | 5. E | | | | |
| | 6. C | | | | |
| 23 | 1. B | Y | N | N | N |
| | 2. F | | | | |
| | 3. D | | | | |
| | 4. C | | | | |
| | 5. E | | | | |
| | 6. A | | | | |
| 24 | 1. F | Y | N | Y | N |
| | 2. C | | | | |
| | 3. E | | | | |
| | 4. D | | | | |
| | 5. A | | | | |
| | 6. B | | | | |
| 25 | 1. D | Y | N | Y | N |
| | 2. C | | | | |
| | 3. B | | | | |
| | 4. F | | | | |
| | 5. E | | | | |
| | 6. A | | | | |
| 26 | 1. C | Y | N | Y | N |
| | 2. D | | | | |
| | 3. F | | | | |
| | 4. A | | | | |
| | 5. E | | | | |
| | 6. B | | | | |
| 27 | 1. A | Y | N | Y | N |
| | 2. F | | | | |
| | 3. E | | | | |
| | 4. D | | | | |
| | 5. B | | | | |
| | 6. C | | | | |
| 28 | 1. B | Y | N | Y | N |
| | 2. E | | | | |
| | 3. C | | | | |
| | 4. F | | | | |
| | 5. A | | | | |
| | 6. D | | | | |
| 29 | 1. F | N | N | N | NA |
| | 2. E | | | | |
| | 3. B | | | | |
| | 4. C | | | | |
| | 5. D | | | | |
| | 6. A | | | | |
| 30 | 1. D | Y | N | Y | N |
| | 2. F | | | | |
| | 3. E | | | | |
| | 4. C | | | | |
| | 5. A | | | | |
| | 6. B | | | | |
| 31 | 1. A | N | N | N | NA |
| | 2. B | | | | |
| | 3. F | | | | |
| | 4. D | | | | |
| | 5. C | | | | |
| | 6. E | | | | |
| 32 | 1. B | Y | Y | Y | N |
| | 2. C | | | | |
| | 3. A | | | | |
| | 4. E | | | | |
| | 5. F | | | | |
| | 6. D | | | | |

Prior to each test day (i.e. the night before), subjects were advised to continue their normal oral hygiene habit but avoid oral hygiene (brushing their teeth) and food intake on the morning of their assessments. On each test day, the breath odour judge carried out breath assessment and the laboratory technician took instrumental measurement. Tongue-scrape samples were removed by the subjects themselves, prior to and following treatment, for microbial recovery of viable count. A washout toothpaste was used by all subjects. With the exception of the six test day mornings, subjects were asked to not alter their normal oral hygiene regime throughout the 6-week study. Tooth brushing, using the washout toothpaste provided, was recommended twice daily for the duration of the trial. On test day mornings, only water was allowed to be consumed and only up to one hour before testing.

All formulations were presented as 10 ml solutions in unlabelled plastic container handed to the subject.

The selected ion flow tube mass spectrometry (SIFT-MS) results for H₂S, volatile organic compounds (VOCs) and volatile sulphur compounds (VSCs) in the exhaled breath of each subject are shown in FIGS. 22, 23 and 24.

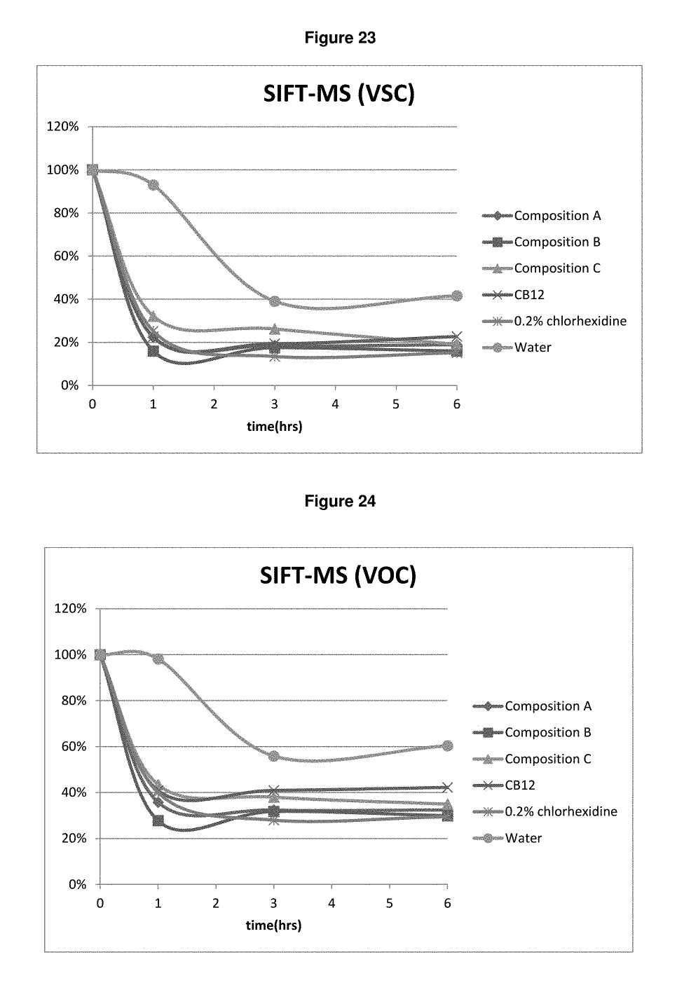

It can be seen from FIG. 22 that Composition A and Composition B of the present invention reduce the amount of hydrogen sulfide in the oral cavity in a similar manner to CB12 or 0.2% chlorhexidine. From FIG. 23 it can be seen that Compositions A and B of the present invention also reduce VSCs in the oral cavity. Both Compositions A and B reduce the % of VSCs more than CB12 and at a comparable level to 0.2% chlorhexidine. FIG. 24 then demonstrates that Compositions A, B and C of the present invention reduce VOCs in the oral cavity in a similar manner to CB12.

The combination of HT61, chlorhexidine and a zinc compound such as zinc acetate thus demonstrates a synergistic effect against hydrogen sulfide, volatile sulfur compounds and volatile organic compounds which is advantageous for patients suffering from oral malodour.

The microbial recovery results from the tongue scrape samples are compared for statistical significance (i.e. P<0.05) below in Tables 20, 21 and 22. Table 20 relates to facultative anaerobes; Table 21 relates to strict anaerobes, and Table 22 contains the total viable count. Facultative anaerobes are organisms that make ATP (adenosine triphosphate) by aerobic respiration if oxygen is present but are capable of switching to fermentation or anaerobic respiration if oxygen is absent. Strict anaerobes can only grow without oxygen. In the presence of oxygen they die.

TABLE 20

| | Composition A | Composition B | Composition C | CB12 | 0.2% CHX | Water |
|---|---|---|---|---|---|---|
| Composition A | | NS | NS | NS | P < 0.05 | P < 0.05 |
| Composition B | | | NS | NS | P < 0.05 | P < 0.05 |
| Composition C | | | | NS | P < 0.05 | P < 0.05 |
| CB12 | | | | | P < 0.05 | P < 0.05 |
| 0.2% CHX | | | | | | P < 0.05 |
| Water | | | | | | |

NS = not significant

TABLE 21

| | Composition A | Composition B | Composition C | CB12 | 0.2% CHX | Water |
|---|---|---|---|---|---|---|
| Composition A | | NS | NS | NS | P < 0.05 | P < 0.05 |
| Composition B | | | NS | NS | P < 0.05 | P < 0.05 |
| Composition C | | | | NS | P < 0.05 | P < 0.05 |
| CB12 | | | | | P < 0.05 | P < 0.05 |
| 0.2% CHX | | | | | | P < 0.05 |
| Water | | | | | | |

TABLE 22

| | Composition A | Composition B | Composition C | CB12 | 0.2% CHX | Water |
|---|---|---|---|---|---|---|
| Composition A | | NS | NS | NS | P < 0.05 | P < 0.05 |
| Composition B | | | NS | NS | P < 0.05 | P < 0.05 |
| Composition C | | | | NS | P < 0.05 | P < 0.05 |
| CB12 | | | | | P < 0.05 | P < 0.05 |
| 0.2% CHX | | | | | | P < 0.05 |
| Water | | | | | | |

All of the formulations tested showed an antimicrobial effect against the biofilm (for facultative and strict anaerobes and total viable count) which was significant compared to water control. CHX (0.2% w/v) showed additional antimicrobial effects, producing low CFU counts compared to all other treatments (P<0.05). The use of 0.2% w/v CHX as an oral composition is, however, not favoured because of the side effects mentioned herein and known in the art including teeth staining. The oral composition of the invention avoids such side effects by including a much lower concentration of chlorhexidine together with HT61 and optionally a zinc compound.

1. An oral composition comprising 4-methyl-8-phenoxy-1-(2-phenylethyl)-2,3-dihydro-1H-pyrrolo[3,2-c]quinoline or a pharmaceutically acceptable salt and/or solvate thereof and about 0.001% w/v to about 0.06% w/v of chlorhexidine or a pharmaceutically acceptable salt and/or solvate thereof, wherein the composition is in the form of a mouthwash.

2. The oral composition according to claim 1, wherein the composition comprises from about 0.01% w/v to about 0.05% w/v of chlorhexidine or a pharmaceutically acceptable salt and/or solvate thereof, preferably from about 0.01% w/v to about 0.03% w/v.

3. The oral composition according to claim 1, wherein the composition comprises from about 0.01% w/v to about 0.03% w/v of 4-methyl-8-phenoxy-1-(2-phenylethyl)-2,3-dihydro-1H-pyrrolo[3,2-c]quinoline or a pharmaceutically acceptable salt and/or solvate thereof.

4. The oral composition according to claim 1, wherein the composition further comprises a zinc compound.

5. The oral composition according to claim 4, wherein the zinc compound is zinc acetate.

6. The oral composition according to claim 1, wherein the composition does not include a surfactant.

7. An oral composition comprising 4-methyl-8-phenoxy-1-(2-phenylethyl)-2,3-dihydro-1H-pyrrolo[3,2-c]quinoline or a pharmaceutically acceptable salt and/or solvate thereof, chlorhexidine or a pharmaceutically acceptable salt and/or solvate thereof, and a zinc compound.

8. The oral composition according to claim 7, wherein the zinc compound is zinc acetate.

9. The oral composition according to claim 7, wherein the composition includes about 0.001% w/v to about 0.06% w/v of chlorhexidine or a pharmaceutically acceptable salt and/or solvate thereof.

10. The oral composition according to claim 1 for use in the prevention and/or treatment of a microbial infection.

11. The oral composition according to claim 1 for use in killing microorganisms associated with a microbial infection.

12-15. (canceled)

16. The method according to claim 20, wherein the infection is a bacterial infection.

17. The method according to claim 20, wherein the infection is an oral bacterial infection.

18. The method according to claim 16, wherein the infection is caused by Staphylococci.

19. The method according to claim 18, wherein the infection is caused by Staphylococcus aureus.

20. A method of treating or preventing a microbial infection in a mammal comprising administering an oral composition comprising 4-methyl-8-phenoxy-1-(2-phenylethyl)-2,3-dihydro-1H-pyrrolo[3,2-c]quinoline or a pharmaceutically acceptable salt and/or solvate thereof, and about 0.001% w/v to about 0.06% w/v of chlorhexidine or a pharmaceutically acceptable salt and/or solvate thereof, or an oral composition comprising 4-methyl-8-phenoxy-1-(2-phenylethyl)-2,3-dihydro-1H-pyrrolo[3,2-c]quinoline or a pharmaceutically acceptable salt and/or solvate thereof, chlorhexidine or a pharmaceutically acceptable salt and/or solvate thereof and a zinc compound, to the mammal, wherein the composition is in the form of a mouthwash.

21-23. (canceled)
